(19) United States
(12) Patent Application Publication
(10) Pub. No.: US 2025/0009917 A1
(43) Pub. Date: Jan. 9, 2025

(54) MACROCYCLE CONTAINING COMPOUNDS AND RADIOLABELLED COMPLEXES THEREOF, AS LIGANDS IN TARGETED RADIOTHERAPY APPLICATIONS

(71) Applicants: THE UNIVERSITY OF MELBOURNE, Victoria (AU); OLIVIA NEWTON-JOHN CANCER RESEARCH INSTITUTE, Victoria (AU)

(72) Inventors: Katherine Anne Morgan, Victoria (AU); Paul Stephen Donnelly, Victoria (AU); Christian Werner Wichmann, Victoria (AU); Andrew Mark Scott, Victoria (AU)

(21) Appl. No.: 18/696,307

(22) PCT Filed: Sep. 29, 2022
(86) PCT No.: PCT/AU2022/051165

§ 371 Date: Mar. 27, 2024

(30) Foreign Application Priority Data
Sep. 29, 2021 (AU) 2021903120

(51) Int. Cl. A61K 51/10; A61K 51/04; A61P 35/00; C07B 59/00

(52) U.S. Cl. CPC A61K 51/1093; A61K 51/0482; A61P 35/00; C07B 59/002; A61K 2121/00; C07B 2200/05

(57) ABSTRACT

The present invention relates to compounds of formula (I) that have the ability to be used as metal complexes in radiotherapy.

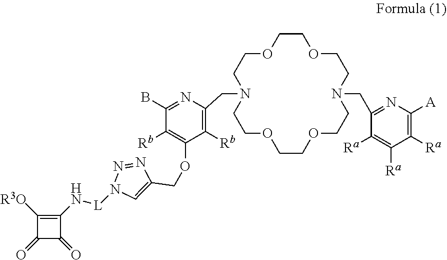

wherein
A is selected from the group consisting of CO2R1, and PO3R1;
B is selected from the group consisting of CO2R2, and PO3R2;
R1, R2 and R3 are each independently selected from the group consisting of H and C1-C12alkyl;
each Ra is independently selected from the group consisting of H, F, Cl, Br, I, CH3, CH2CH3, CH(CH3)2, OH, OCH3, OCH2CH3, CF3, OCF3, NO2, NH2, and CN;
each Rb is independently selected from the group consisting of H, F, Cl, Br, I, CH3, CH2CH3, CH(CH3)2, OH, OCH3, OCH2CH3, CF3, OCF3, NO2, NH2, and CN;
L is a linker having from 1 to 20 atoms in the normal chain;
or a pharmaceutically acceptable salt thereof.

### TECHNICAL FIELD

The present invention relates to macrocycle containing compounds that can be used as ligands in targeted radiotherapy applications. The invention also relates to certain metal complexes of these compounds as well as their method of use.

### BACKGROUND OF INVENTION

In the last 200 years there has been significant progress made in medical research such that average life expectancy has dramatically increased as effective medical treatments have been developed for a significant number of diseases/conditions. Unfortunately, this success in providing improved treatments for certain conditions has meant that humans are now living long enough to develop and suffer from other conditions that were previously relatively rare as they are conditions that typically develop very slowly or a disease of the “aged”.

For example, it is estimated that cancer is the largest contribution (16%) to the burden of disease in Australia and is the second-leading cause of death world-wide, with an estimated 10 million people dying from cancer in 2020, according to the World Health Organization although this number is likely to be significantly lower than the total actual number due to potential under reporting in less developed countries. Nevertheless, it is estimated that in 2020 there were 1.8 million deaths from lung cancer, 935,000 deaths from colon and rectal cancer, 830,000 deaths from liver cancer, 769,000 deaths from stomach cancer and 685,000 deaths from breast cancer highlighting the extent of the problem. The economic impact of cancer is therefore significant and is increasing. The global annual economic cost associated with cancer is estimated to be in excess of $US 1.2 trillion although the exact cost is hard to quantify. This figure is expected to rise as life expectancy increases and as lifestyle, diet and/or environmental factors change over time.

Cancer is a rather generic term used to describe a large group of diseases that can impact upon any port of the body. A feature common to almost all cancers is the rapid creation by the body of abnormal cells that grow beyond their normal boundaries and which can then invade other parts of the body. Cancer can therefore be described as an uncontrolled proliferation of cells, which can invade and spread to other sites of the body. The causes of cancer are generally attributed to environmental or genetic factors. More than 100 different types of cancer are known, with more new types characterised each year.

Cancer cells can exist in a number of different forms. For example, they may exist as a solid tumour, in which the cancer cells are massed together, or dispersed, as in leukemia. Cancer cells are often referred to as “malignant”, because they divide endlessly, eventually crowding out nearby cells and spreading to other parts of the body. The tendency of cancer cells to spread from one organ to another or from one part of the body to another distinguishes them from benign tumour cells, which overgrow but do not spread to other organs or parts of the body. Malignant cancer cells eventually metastasize and spread to other parts of the body via the bloodstream or lymphatic system, where they can multiply and form new tumours. This sort of tumour progression makes cancer a deadly disease.

Although there have been great improvements in the diagnosis and treatment of cancer, many people still die from cancer each year. Accordingly, there have been a number of treatments developed that have been used to treat cancer patients with a persistent drive to develop further and improved cancer treatments.

One area of growing interest is the use of radiotherapy in the treatment of cancers. Radiotherapy is based on the observation that, at high does, radiation kills cancer cells or slows their growth as a result of the radiation damaging the DNA of the cancer cells. Cancer cells, like other cells, whose DNA is damaged beyond repair stop dividing and die. Following cell death, the cells are broken down and removed from the body.

Radiation therapy is typically divided into two main types namely (1) external beam radiation therapy and (2) internal radiotherapy and the type of radiotherapy used in any case will depend upon ta number of factors specific to the patient.

External beam radiotherapy involves the use of a machine that aims radiation at the cancer on a patient. This type of radiotherapy aims to treat a specific part of the body such that only the area in which the cancer is located will be subjected to radiation rather than your whole body. Whist this type of therapy can be very successful in the case of tumours that are located deep within the body there is the necessity that some healthy cells are exposed to the radiation as it targets the cancer cells within the body.

Internal radiotherapy comprises either the seeding of a radioactive substance at the site of the tumour or the use of a chemotherapeutic agent that “targets” the site of the cancer. In these targeted approaches a ligand for the radionuclide is typically conjugated to an antibody that targets the specific cancer to be treated. For example, targeted alpha particle therapy has been developed for the treatment of soft tissue metastases. In these therapies lethal alpha particle emitting radionuclides are conjugated to tumour targeting vectors using chelators. This therapy allows for the delivery of cytotoxic levels of alpha radiation to be selectively delivered to cancer calls.

As a result, there has been significant research interest aimed at developing conjugates that can be used to bind an alpha particle emitting radionuclide and which can then further be elaborated to include a cancer targeting moiety. A number of potential conjugates have been developed.

One of the best conjugates currently employed is H₄DOTA (1).

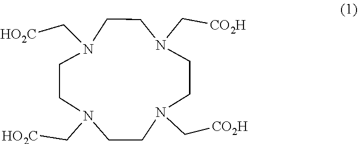 [SMILES: O=C(O)CN1CCN(CC(=O)O)CCN(CC(=O)O)CCN(CC(=O)O)CC1]

This conjugate is relatively easy to access but suffers from the observation that the thermodynamic stability of complexes of H₄DOTA decrease as the ionic metal ion increases with the result that it is not ideally suited for all metal complexes such as Ac³⁺ which is the largest plus 3 (trivalent) ion in the periodic table. In addition, the radiolabelling kinetics of this ligand are relatively slow with some radionuclides (such as Ac³⁺) leading to the requirement for labelling at elevated temperatures if short labelling times are required.

As a result, there have been a number of studies directed towards the development of alternative conjugates. One alternative conjugate that has been developed is N,N′-bis[(6-carboxy-2-pyridil)methyl]-4,13-diaza-18-crown-6 (2) which is colloquially known as H₂macropa.

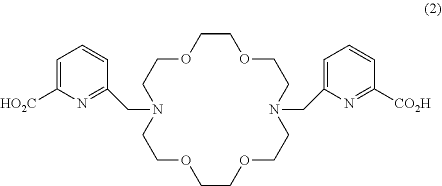 [SMILES: O=C(O)c1cccc(CN2CCOCCOCCN(Cc3cccc(C(=O)O)n3)CCOCCOCC2)n1]

Another conjugate that has been developed is an analogue of H₂macropa. Namely H₂macropa-NCS (3):

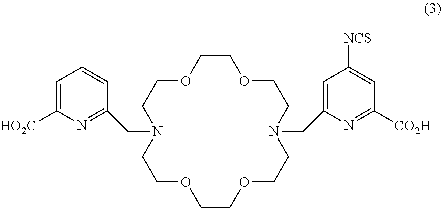

Whilst these conjugates were better able to be radiolabelled without the need for elevated temperatures they were not without problems as it was observed that aggregation of the conjugate occurred.

As such, there is significant interest in developing further compounds that could be used as radionuclide conjugates in targeted therapy.

### SUMMARY OF THE INVENTION

The present applicants have conducted research aimed at developing compounds that could be used as potential radionuclide conjugates and which may therefore find application in targeted therapy.

As a result of these studies the applicants have identified compounds show significant potential as conjugates for radionuclides.

Accordingly, in one embodiment the present invention provides a compound of Formula (I):

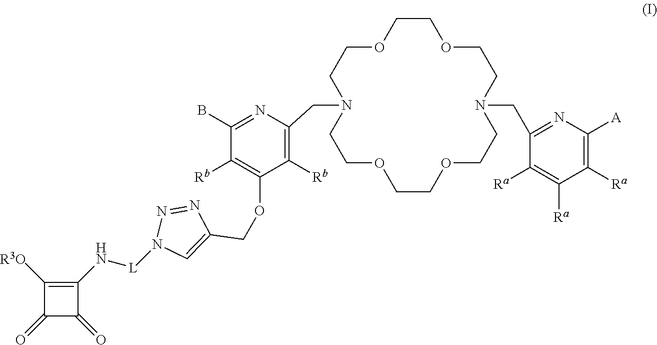

wherein
A is selected from the group consisting of CO₂R¹, and PO₃R¹;
B is selected from the group consisting of CO₂R², and PO₃R²;
R¹, R² and R³ are each independently selected from the group consisting of H and C₁-C₁₂alkyl;
each R^{a} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
each R^{b} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
L is a linker having from 1 to 20 atoms in the normal chain;
or a pharmaceutically acceptable salt thereof.

The compounds of the present invention have been found to have the ability to form metal complexes with radionuclides which can then be used in targeted therapies.

In yet an even further aspect the present invention provides a compound of Formula (Ia):

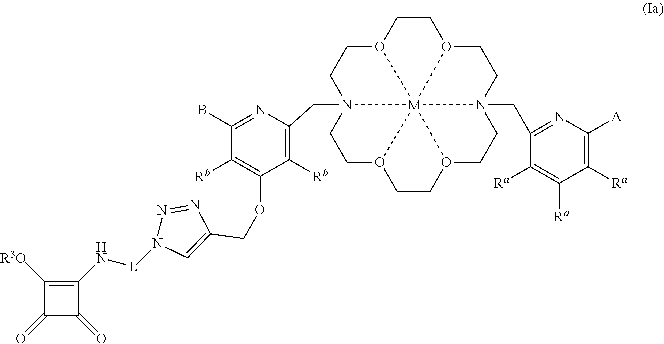

wherein
A is selected from the group consisting of CO₂R¹, and PO₃R¹;
B is selected from the group consisting of CO₂R², and PO₃R²;
R¹, R² and R³ are each independently selected from the group consisting of H and C₁-C₁₂alkyl;
each R^{a} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
each R^{b} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
L is a linker having from 1 to 20 atoms in the normal chain;
M is a radionuclide;
or a pharmaceutically acceptable salt thereof.

The compounds of formula (I) or (Ia) described herein have the ability to react with antibodies to form a targeting conjugate. Accordingly, in yet an even further aspect the present invention provides a compound of formula (Ib):

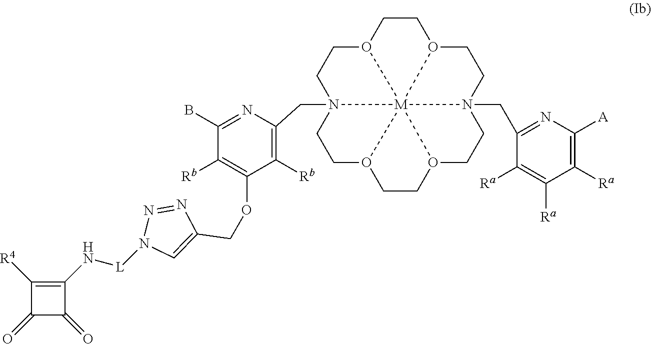

wherein
A is selected from the group consisting of CO₂R¹, and PO₃R¹;
B is selected from the group consisting of CO₂R², and PO₃R²;
R¹, and R² are each independently selected from the group consisting of H and C₁-C₁₂alkyl;
R⁴ is a monoclonal antibody;
each R^{a} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
each R^{b} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
L is a linker having from 1 to 20 atoms in the normal chain;
M is a radionuclide;
or a pharmaceutically acceptable salt thereof.

In yet an even further aspect the present invention provides a method for the synthesis of a compound of formula (I) as described above the method comprising:
(a) providing a compound of formula (10)

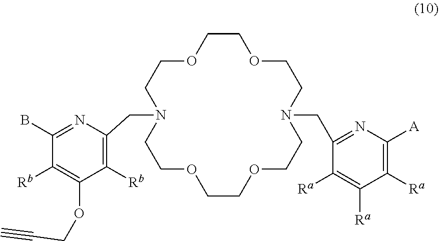

A is selected from the group consisting of CO₂R¹, and PO₃R¹;
B is selected from the group consisting of CO₂R², and PO₃R²;
R¹, and R² are each independently selected from the group consisting of H and C₁-C₁₂alkyl;
each R^{a} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
each R^{b} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
(b) reacting the compound of formula (10) with a compound of formula (11)

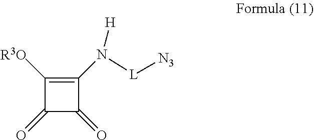

where R³ is selected from the group consisting of H and C₁-C₁₂alkyl;
L is a linker having from 1 to 20 atoms in the normal chain;
in the presence of a copper(I) catalyst.

In yet an even further aspect the present invention provides a pharmaceutical composition comprising a compound according to Formula (I), Formula (Ia) or Formula (Ib) and a pharmaceutically acceptable diluent, excipient or carrier.

In yet an even further aspect the present invention provides a method of treating a subject comprising administering a therapeutically effective amount of a compound of Formula (Ib) to the subject. In certain embodiments the subject is suffering from cancer.

### BRIEF DESCRIPTION OF THE DRAWINGS

FIG. 1. TLC analysis of free Ac-225 using 0.4 M sodium citrate pH 4+10% methanol (origin is at 15 mm, solvent front at 80 mm; free Ac-225 is at solvent front: R_{f}=0.8−1.0).

FIG. 2. iTLC analysis of free Ac-225 using 50 mM citric acid pH 5 (origin is at 15 mm, solvent front at 80 mm; free Ac-225 is at solvent front: R_{f}=0.8-1.0).

FIG. 3. TLC analysis of crude [²²⁵Ac]Ac-macropa at 10⁻³ M chelator concentration using 0.4 M sodium citrate pH 4+10% methanol (origin is at 10 mm, solvent front at 80 mm; [²²⁵Ac]Ac-macropa remains at the origin: R_{f}=0; free Ac-225 is at solvent front: R_{f}=0.8−1.0).

FIG. 4. Summary of radiochemical yields of various Ac-225 labelled chelators at different chelator concentrations incubated for 2 hours at ambient temperature unless noted otherwise. Radiochemical yields determined via TLC analysis of crude reaction mixtures using 0.4 M sodium citrate pH 4+10% methanol.

FIG. 5. Summary of radiochemical yields of [²²⁵Ac]Ac-macropa-tzPEG₃SqOEt over time and at different chelator concentrations incubated at ambient temperature. Radiochemical yields determined via TLC analysis of crude reaction mixtures using 0.4 M sodium citrate pH 4+10% methanol.

FIG. 6. Summary of radiochemical yields of [²²⁵Ac]Ac-DOTA-methyltetrazine over time and at different chelator concentrations incubated at 90° C. Radiochemical yields determined via TLC analysis of crude reaction mixtures using 0.4 M sodium citrate pH 4+10% methanol.

FIG. 7. iTLC analyses of (a) [²²⁵Ac]Ac-macropa-tzPEG₃Sq-EGFRVIII IgG1 and (b) [²²⁵Ac]Ac-macropa-tzPEG₃Sq-EphA3 IgG1 at 10⁻⁶ M antibody concentration incubated for 15 minutes at ambient temperature (50 mM citric acid pH 5; origin is at 10 mm, solvent front at 80 mm; [²²⁵Ac]Ac-macropa-tzPEG₃Sq-conjugated monoclonal antibodies remain at the origin: R_{f}=0; free Ac-225 is at solvent front: R_{f}=0.8-1.0).

FIG. 8. SE-HPLC chromatograms of H₂macropa-tzPEG₃Sq-immunoconjugates (UV₂₈₀).

FIG. 9. SE-HPLC chromatograms of [²²⁵Ac]Ac-macropa-tzPEG₃Sq-immunoconjugates (UV₂₈₀ and radiation).

FIG. 10. Serum stability study of [²²⁵Ac]Ac-macropa-tzPEG₃Sq- and [²²⁵Ac]Ac-DOTA-dhPzPEG₄-conjugated monoclonal antibodies at different chelator-antibody-ratios; (a) Radiochemical purities determined using iTLC (50 mM EDTA pH 5); (b) Immunoreactivity determined with U87MG.de2-7 and U251 cells for EGFRVIII IgG1 and EphA3 IgG1, respectively.

FIG. 11. (a) Tumour growth curves and (b) Kaplan Meier survival curves of U251 murine xenografts (n=5) treated with varying amounts of [²²⁵Ac]Ac-macropa-tzPEG₃Sq-EphA3 IgG1 (0.25-1.0 μg/9.25-37.0 kBq) or vehicle.

FIG. 12. (a) Tumour growth curves and (b) Kaplan Meier survival curves of U87MG.de2-7 murine xenografts (n=5) treated with varying amounts of [²²⁵Ac]Ac-macropa-tzPEG₃Sq-EGFRVIII IgG1 (0.25-1.0 μg/9.25-37.0 kBq) or vehicle.

### DETAILED DESCRIPTION

In this specification a number of terms are used that are well known to a skilled addressee. Nevertheless, for the purposes of clarity a number of terms will be defined.

In the definitions of a number of substituents below it is stated that “the group may be a terminal group or a bridging group”. This is intended to signify that the use of the term is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present application no such distinction is made and most groups may be either a bridging group or a terminal group.

“Alkyl” as a group or part of a group refers to a straight or branched aliphatic hydrocarbon group, preferably a C₁-C₁₂ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

The term “normal chain” refers to the direct chain joining the two ends of a linking moiety.

The term “pharmaceutically acceptable salts” refers to salts that retain the desired biological activity of the above-identified compounds and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propanoic, succinic, glycolic, gluconic, lactic, malic, tartaric, citric, fumaric, maleic, alkyl sulfonic, arylsulfonic. In a similar vein base addition salts may be prepared by ways well known in the art using organic or inorganic bases. Example of suitable organic bases include simple amines such as methylamine, ethylamine, triethylamine and the like. Examples of suitable inorganic bases include NaOH, KOH, and the like. Additional information on pharmaceutically acceptable salts can be found in Remington's Pharmaceutical Sciences, 19th Edition, Mack Publishing Co., Easton, PA 1995. In the case of agents that are solids, it is understood by those skilled in the art that the inventive compounds, agents and salts may exist in different crystalline or polymorphic forms, all of which are intended to be within the scope of the present invention and specified formulae.

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state.

As stated above the compounds of the invention have the formula (I), (Ia) or (Ib). As with any group of structurally related compounds which possess a particular utility, certain embodiments of variables of the compounds of the formula (I), (Ia) or (Ib). are particularly useful in their end use application.

In the compounds of the invention A is selected from the group consisting of CO₂R¹, and PO₃R¹. In some embodiments A is CO₂R¹. In some embodiments A is PO₃R¹.

In the compounds of the invention B is selected from the group consisting of CO₂R², and PO₃R². In some embodiments B is CO₂R². In some embodiments B is PO₃R².

In the compounds of the invention R¹, R² and R³ are each independently selected from the group consisting of H and C₁-C₁₂alkyl.

In some embodiments R¹ is H. In some embodiments R¹ is C₁-C₁₂alkyl. In some embodiments R¹ is selected from the group consisting of methyl, ethyl, isopropyl, propyl, 2-ethyl-propyl, 3,3-dimethyl-propyl, butyl, isobutyl, 3,3-dimethyl-butyl, 2-ethyl-butyl, pentyl, 2-methyl, pentyl, and hexyl.

In some embodiments R² is H. In some embodiments R² is C₁-C₁₂alkyl. In some embodiments R² is selected from the group consisting of methyl, ethyl, isopropyl, propyl, 2-ethyl-propyl, 3,3-dimethyl-propyl, butyl, isobutyl, 3,3-dimethyl-butyl, 2-ethyl-butyl, pentyl, 2-methyl, pentyl, and hexyl.

In some embodiments of the compounds of the invention A is CO₂R¹, B is CO₂R², R¹ is H and R² is H. This provides compounds of formula (II), (IIa) and (IIb) respectively:

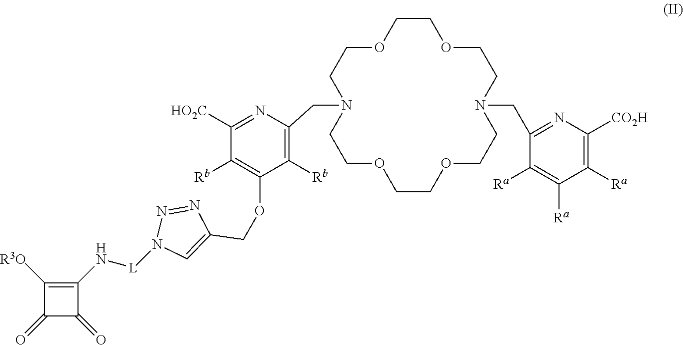

wherein R^{a}, R^{b}, L and R³ are as defined above.

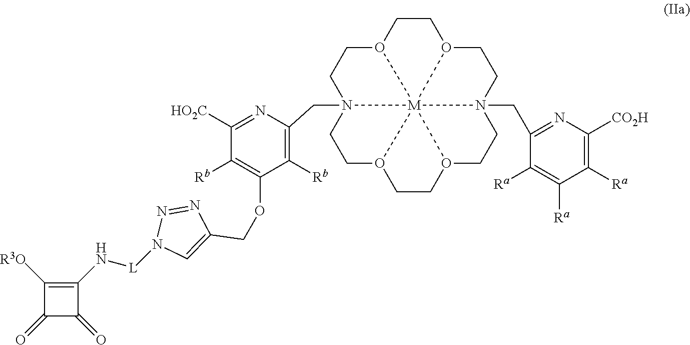

wherein R^{a}, R^{b}, L R³ and M are as defined above.

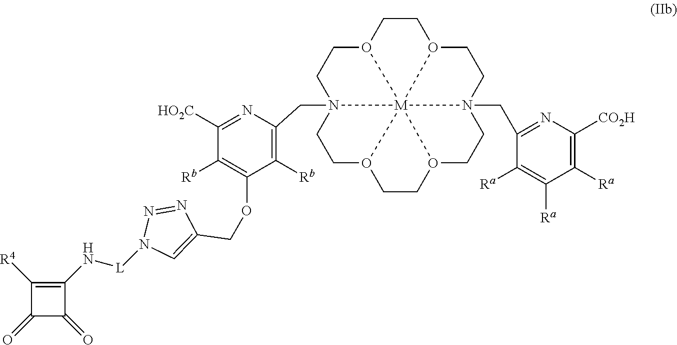

wherein R^{a}, R^{b}, L R⁴ and M are as defined above.

In the compounds of the invention each R^{a} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN. In some embodiments each R^{a} is independently selected from H, F, Cl, Br, I, CH₃, CH₂CH₃, OH, OCH₃, OCH₂CH₃, and CN. In some embodiments each R^{a} is independently selected from H, CH₃, CH₂CH₃, and OH. In some embodiments each R^{a} is H.

In the compounds of the invention each R^{b} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN. In some embodiments each R^{b} is independently selected from H, F, Cl, Br, I, CH₃, CH₂CH₃, OH, OCH₃, OCH₂CH₃, and CN. In some embodiments each R^{b} is independently selected from H, CH₃, CH₂CH₃, and OH. In some embodiments each R^{b} is H.

In some embodiments of the compounds of the invention A is CO₂R¹, B is CO₂R², R¹ is H, R² is H, each R^{a} is H and each R^{b} is H. This provides compounds of formula (III), (Ilila) and (IIIb) respectively.

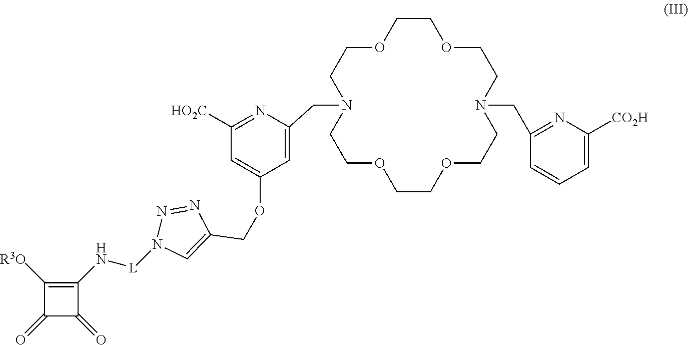

wherein L and R³ are as defined above.

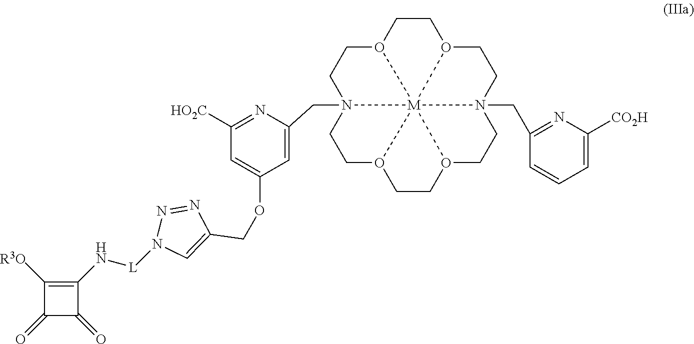

wherein L R³ and M are as defined above.

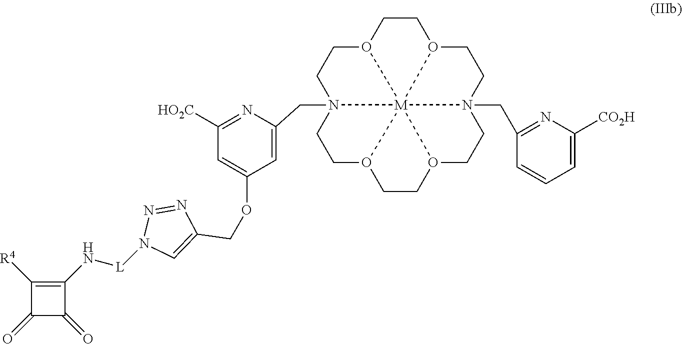

wherein L, R⁴ and M are as defined above.

In some embodiments R³ is H. In some embodiments R³ is C₁-C₁₂alkyl. In some embodiments R³ is selected from the group consisting of methyl, ethyl, isopropyl, propyl, 2-ethyl-propyl, 3,3-dimethyl-propyl, butyl, isobutyl, 3,3-dimethyl-butyl, 2-ethyl-butyl, pentyl, 2-methyl, pentyl, and hexyl. In some embodiments R³ is methyl. In some embodiments R³ is ethyl.

In the compounds of the invention containing an R⁴ moiety, then R⁴ is an antibody. In certain embodiments R⁴ is a monoclonal antibody. In certain embodiments R⁴ is selected from the group consisting of Penpulimab, Sintilimab, Toripalimab, Omburtamab, Tisotumab, Retifanlimab, Ublituximab, Anifrolumab, Loncastuximab, Balstilimab, Dostarlimab, Oportuzumab, Margetuximab, Naxitamab, Belantamab, Tafasitamab, Sacituzumab, Isatuximab, Trastuzumab (Herceptin), Girentuximab, Ifabotuzumab, Depatuxizumab, Enfortumab, Polatuzumab, Emapalumab, Cemiplimab, Moxetumomab, Mogamulizumab, Durvalumab, Avelumab, Atezolizumab, Olaratumab, Daratumumab, Elotuzumab, Necitumumab, Dinutuximab, Nivolumab, Blinatumomab, Blinatumomab, Ramucirumab, Obinutuzumab, Ado-trastuzumab, Pertuzumab, Brentuximab, Ipilimumab, Ofatumumab, Catumaxomab, Panitumumab, Bevacizumab, Cetuximab, Tositumomab-1131, Ibritumomab, Alemtuzumab, Gemtuzumab, Rituximab, Edrecolomab, Nimotuzumab, Prolgolimab, and Cetuximab.

In one embodiment the antibody is Herceptin. In one embodiment the antibody is an EphA3 IgG1 monoclonal antibody. In one embodiment the antibody is an EGFRVIII IgG1 monoclonal antibody. In one embodiment the antibody is an isotype control IgG1.

In the compounds of the invention L is a linker having from 1 to 20 atoms in the normal chain. In some embodiments L is a linker having from 2 to 19 atoms in the normal chain. In some embodiments L is a linker having from 3 to 18 atoms in the normal chain. In some embodiments L is a linker having from 4 to 17 atoms in the normal chain. In some embodiments L is a linker having from 5 to 16 atoms in the normal chain. In some embodiments L is a linker having from 6 to 15 atoms in the normal chain. In some embodiments L is a linker having from 7 to 14 atoms in the normal chain. In some embodiments L is a linker having from 8 to 13 atoms in the normal chain. In some embodiments L is a linker having from 9 to 12 atoms in the normal chain.

In some embodiments L is a linker having 1 atom in the normal chain. In some embodiments L is a linker having 2 atoms in the normal chain. In some embodiments L is a linker having 3 atoms in the normal chain. In some embodiments L is a linker having 4 atoms in the normal chain. In some embodiments L is a linker having 5 atoms in the normal chain. In some embodiments L is a linker having 6 atoms in the normal chain. In some embodiments L is a linker having 7 atoms in the normal chain. In some embodiments L is a linker having 8 atoms in the normal chain. In some embodiments L is a linker having 9 atoms in the normal chain. In some embodiments L is a linker having 10 atoms in the normal chain. In some embodiments L is a linker having 11 atoms in the normal chain. In some embodiments L is a linker having 12 atoms in the normal chain. In some embodiments L is a linker having 13 atoms in the normal chain. In some embodiments L is a linker having 14 atoms in the normal chain. In some embodiments L is a linker having 15 atoms in the normal chain. In some embodiments L is a linker having 16 atoms in the normal chain. In some embodiments L is a linker having 17 atoms in the normal chain. In some embodiments L is a linker having 18 atoms in the normal chain. In some embodiments L is a linker having 19 atoms in the normal chain. In some embodiments L is a linker having 20 atoms in the normal chain.

In some embodiments L is a linker having the formula:

—(CH₂)ₘ—

here m is an integer elected from the group consisting of 1, 2, 3, 4, 5, 6, 7, 8, 9 10, 11, 12, 13, 14, 15, 16, 17, 18, 19, and 20.

In some embodiments L is selected from the group consisting of —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₁₄—, and —(CH₂)₁₅—.

In some embodiments L is a linker of the formula:

—(CH₂CH₂O)ₙ—CH₂CH₂—

wherein n is an integer from the group consisting of 0, 1, 2, 3, 4, and 5.

In some embodiments L is selected from the group consisting of —(CH₂CH₂O)—CH₂CH₂—, —(CH₂CH₂O)₂—CH₂CH₂—, —(CH₂CH₂O)₃—CH₂CH₂—, —(CH₂CH₂O)₄—CH₂CH₂— and —(CH₂CH₂O)₅—CH₂CH₂—.

In some embodiments L is —(CH₂CH₂O)—CH₂CH₂—. In some embodiments L is —(CH₂CH₂O)₂—CH₂CH₂—. In some embodiments L is —(CH₂CH₂O)₃—CH₂CH₂—. In some embodiments L is —(CH₂CH₂O)₄—CH₂CH₂—. In some embodiments L is —(CH₂CH₂O)₅—CH₂CH₋₂.

In a particularly preferred embodiment L is —(CH₂CH₂O)₃—CH₂CH₂—.

The macrocycles of the invention can in principle bind a number of metals. In application the compounds are used in radiotherapy and as such it is preferred that M is a radionuclide. In certain embodiments M is selected from the group consisting of Actinium-225, Lutetium-177, Zirconium-89, Terbium-149, Terbium-152, Terbium-155, Terbium-161, Radium-223, Bismuth-212, Indium-111, Yttrium-86, Yttrium-89, Yttrium-90, and Lead-212.

In some embodiments M is Actinium-225. In some embodiments M is Lutetium-177. In some embodiments M is Zirconium-89. In some embodiments M is Terbium-149. In some embodiments M is Terbium-152. In some embodiments M is Terbium-155. In some embodiments M is Terbium-161. In some embodiments M is Radium-223. In some embodiments M is Bismuth-212. In some embodiments M is Indium-111. In some embodiments M is Yttrium-86. In some embodiments M is Yttrium-89. In some embodiments M is Yttrium-90. In some embodiments M is Lead-212.

In a particularly preferred embodiment M is Actinium-225.

The compounds of the invention which contain a radionuclide M are typically formed by incubation of the metal with the conjugate of the appropriate formula. The incubation may occur at any temperature although it is found that the binding of radionuclides with the compounds of the present invention is very rapid such that the binding can be carried out at room temperature and is still quite rapid. Once bound the complex thus formed is found to be very stable.

In principle the metal can be complexed either with the compound containing an antibody although it is typically found to be more efficient to complex the radionuclide with the compound prior to addition of the antibody. Without wishing to be bound by theory it is felt that this ensure more efficient complexation as sites on the antibody may compete with the macrocycle to bind to the metal. Accordingly, it is usual to react the complex with the radionuclide prior to addition of the antibody.

The compound containing the antibody are typically formed by reaction of a compound of the formula (I), (Ia), (II), (IIa), (III) and (IIIa) with a pendant amine group on the antibody to form the compounds containing an antibody. As will be appreciated by a skilled worker in the field many antibodies contain multiple amine residues available for reactivity with the squaramide moieties on the compounds of the invention. As such in some embodiments each antibody is conjugated to more than one compound of the invention. In some embodiments each antibody is conjugated to 2 compounds of the invention. In some embodiments each antibody is conjugated to 3 compounds of the invention. In some embodiments each antibody is conjugated to 4 compounds of the invention. In some embodiments each antibody is conjugated to 5 compounds of the invention. Control of the reaction stoichiometry and general reaction conditions can be used to control the formation of the final antibody conjugate.

The compounds of the invention containing a radionuclide (M) and a targeting antibody (R⁴) have the ability to be used in the targeted radiotherapy of cancer. As a result of the presence of the antibody on the compound the compounds target (ie selectively bind to) cancer cells. As a result of the selective binding of the compound to the cancer cells the radiation provided by the radionuclide is released in close proximity to the cancer cell thus causing greater damage to the cancer cell rather than the other cells of the body. This is found to be particularly effective when the radionuclide emits alpha particles.

Accordingly compounds of the invention would be expected to have useful therapeutic properties in the treatment of cancers. Examples of cancers include prostate cancer, breast cancer, pancreas cancer, colonic cancer, non-small cell lung cancer, hepatocellular carcinoma, intrahepatic cholangiocarcinoma, renal cell carcinoma, endometrial carcinoma, oesophageal carcinoma, carcinoma of the oesophagus/gastro-oesophageal junction, osteosarcoma, Wilms tumour, mesothelioma, squamous cell carcinoma, glioblastoma multiforme, melanoma and ovarian carcinoma.

Administration of compounds of the invention to humans can be by any of the accepted modes for parenteral administration such as subcutaneous, intramuscular, intravenous and intradermal routes. Injection can be bolus or via constant or intermittent infusion. The active compound is typically included in a pharmaceutically acceptable carrier or diluent and in an amount sufficient to deliver to the patient a therapeutically effective dose.

The compounds of the invention can be administered in any form or mode which makes the compound available for binding to the desired target cell. One skilled in the art of preparing formulations can readily select the proper form and mode of administration depending upon the particular characteristics of the compound selected, the condition to be treated, the stage of the condition to be treated and other relevant circumstances. We refer the reader to Remingtons Pharmaceutical Sciences, 19^{th} edition, Mack Publishing Co. (1995) for further information.

The compounds of the present invention can be administered alone or in the form of a pharmaceutical composition in combination with a pharmaceutically acceptable carrier, diluent or excipient. The compounds of the invention, while effective themselves, are typically formulated and administered in the form of their pharmaceutically acceptable salts as these forms are typically more stable, more easily crystallised and have increased solubility.

The compounds are, however, typically used in the form of pharmaceutical compositions which are formulated depending on the desired mode of administration. As such in some embodiments the present invention provides a pharmaceutical composition including a compound of the invention and a pharmaceutically acceptable carrier, diluent or excipient. The compositions are prepared in manners well known in the art.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

The amount of compound administered will preferably treat and reduce or alleviate the condition. A therapeutically effective amount can be readily determined by an attending diagnostician using conventional techniques and by observing results obtained under analogous circumstances. In determining the therapeutically effective amount a number of factors are to be considered including but not limited to, the species of animal, its size, age and general health, the specific condition involved, the severity of the condition, the response of the patient to treatment, the particular compound administered, the mode of administration, the bioavailability of the preparation administered, the dose regime selected, the use of other medications and other relevant circumstances.

A preferred dosage will be a range from about 0.01 to 300 mg per kilogram of body weight per day. A more preferred dosage will be in the range from 0.1 to 100 mg per kilogram of body weight per day, more preferably from 0.2 to 80 mg per kilogram of body weight per day, even more preferably 0.2 to 50 mg per kilogram of body weight per day. A suitable dose can be administered in multiple sub-doses per day.

### Synthesis of Compounds of the Invention

The compounds of the present invention may be prepared using known organic synthesis techniques and can be synthesized according to any of numerous possible synthetic routes including the reaction routes and synthesis schemes as described below, employing the techniques available in the art using starting materials that are readily available. The preparation of compounds of the embodiments is described in detail in the following examples, but the artisan will recognize that the chemical reactions described may be readily adapted to prepare other agents of the various embodiments.

The reactions for preparing compounds of the invention can be carried out in suitable solvents, which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T.W. Greene and P.G.M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high-performance liquid chromatography (HPLC) or thin layer chromatography.

The expressions, “ambient temperature,” “room temperature,” and “r.t.”, as used herein, are understood in the art, and refer generally to a temperature, e.g. a reaction temperature, that is about the temperature of the room in which the reaction is carried out, for example, a temperature from about 20° C. to about 30° C.

In yet an even further aspect the present invention provides a method for the synthesis of a compound of formula (I):

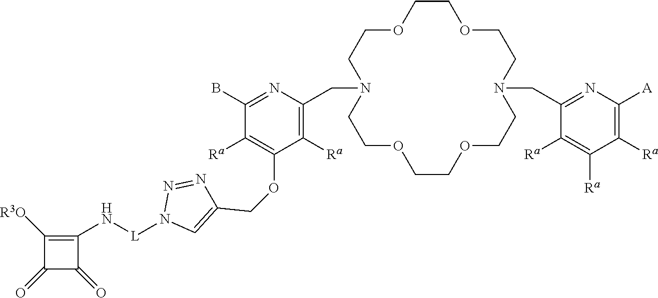

wherein
A is selected from the group consisting of CO₂R¹, and PO₃R¹;
B is selected from the group consisting of CO₂R², and PO₃R²;
R¹, R² and R³ are each independently selected from the group consisting of H and C₁-C₁₂alkyl;
each R^{a} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
each R^{b} is independently selected from the group consisting of H, F, Cl, Br, I, CH₃, CH₂CH₃, CH(CH₃)₂₀H, OCH₃, OCH₂CH₃, CF₃, OCF₃, NO₂, NH₂, and CN;
L is a linker having from 1 to 20 atoms in the normal chain;
or a pharmaceutically acceptable salt thereof;
the method comprising;
(a) providing a compound of formula (II):

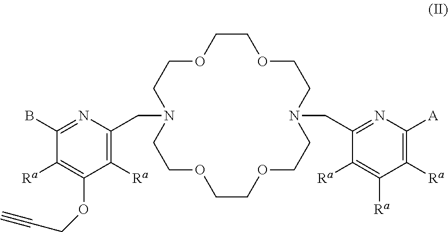

wherein A is selected from the group consisting of CO₂R¹, and PO₃R¹;
B is selected from the group consisting of CO₂R², and PO³R²;
R¹, and R² are each independently selected from the group consisting of H and C₁-C₁₂alkyl;
each R^{a} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
each R^{b} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
(b) reacting the compound of formula (II) with a compound of formula (III):

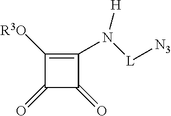

where R³ is selected from the group consisting of H and C₁-C₁₂alkyl;
L is a linker having from 1 to 20 atoms in the normal chain;
in the presence of a copper(I) catalyst.

The reaction may be carried out in any suitable solvent. Examples of suitable solvents include polar organic solvents and combinations thereof. Examples of polar solvents that may be used include dimethyl formamide (DMF), alcohols (such as methanol, ethanol, propanol and t-butanol), dimethyl sulfoxide, tetrahydrofuran and acetonitrile. In some embodiments the solvent is a combination of a polar organic solvent as discussed above and water. In some embodiments the ratio of polar organic solvent to water is from 10:1 to 1:1. In one embodiment the ratio of polar organic solvent to water is from 8:1 to 1:1. In one embodiment the ratio of polar organic solvent to water is from 6:1 to 1:1. In one embodiment the ratio of polar organic solvent to water is from 5:1 to 3:1. In one embodiment the ratio of polar organic solvent to water is about 4:1. In one preferred embodiment the solvent is DMF:water in a volume ratio of 4:1.

The reaction may be carried out across a wide range of temperatures. In some embodiments the reaction is carried out at a temperature from 5° C. to 50° C. In one embodiment the reaction is carried out at a temperature of from 10° C. to 30° C. In one embodiment the reaction is carried out at a temperature of from 15° C. to 25° C. In one embodiment the reaction is carried out at a temperature of from 20° C. to 25° C.

The reaction is typically carried out for a period of time sufficient to achieve complete reaction of the starting materials. In some embodiments the reaction is conducted from 1 to 24 hours. In some embodiments the reaction is conducted from 4 to 20 hours. In some embodiments the reaction is conducted from 8 to 16 hours. In some embodiments the reaction is conducted from 10 to 14 hours.

As stated above the reaction is carried out in the presence of a copper(I) catalyst. Examples of suitable copper catalysts include copper(I) salts (iodide, bromide, chloride, acetate), copper(I) complexes: such as [Cu(CH₃CN)₄]PF₆ and [Cu(CH₃CN)₄]BF₄ or triflate counterion, copper(II) sulfate penta hydrate and copper(II) acetate In one embodiment the copper catalyst is generated in situ by reduction of copper sulfate.

In certain embodiments the reaction is carried out in the presence of a copper(I) ligand that helps to stabilise the copper(I) in solution wherein the copper(I) ligand is selected from the group consisting of TBTA, TEOTA, THPTA, BTTES, BTTAA, BTTP, BTTPS, (BimH)₃, (Bth)₃, BPS, and 4.4′-dimethy;-2,2′-bypyrimidine.

Following completion of the reaction the reaction medium is worked up in way known in the art and the resulting reaction product purified to provide the desired end products.

The invention will now be illustrated by way of examples; however, the examples are not to be construed as being limitations thereto. Additional compounds, other than those described below, may be prepared using methods and synthetic protocols or appropriate variations or modifications thereof, as described herein.

### EXAMPLES

### Experimental

### Materials and Reagents

All solvents and reagents were purchased from standard commercial suppliers and were used as received.

### Instrumentation

¹H, ¹³C, COSY, HSQC, HMBC were all recorded using a Varian FT-NMR 400 of FT NMR 500 spectrometer (Varian, California, USA). All ¹H NMR spectra were acquired at 400 MHz or 500 MHz and ¹³C spectra were acquired at 101 MHz or 126 MHz. The reported peaks were all referenced to solvent peaks in the order of parts per million at 25° C.

ESI-QTOF MS was collected on an Exactive Plus Orbitrap Infusion mass spectrometer (Exactive Series, 2.8 Build 268801, ThermoFisher Scientific). Analysis was performed using Xcalibur 4.0.27.10 (ThermoFisher Scientific).

Protein samples were analysed on Agilent 6220 ESI-TOF LC/MS Mass Spectrometer coupled to an Agilent 1200 LC system (Agilent, Palo Alto, CA). All data were acquired, and reference mass corrected via a dual-spray electrospray ionisation (ESI) source. Acquisition was performed using the Agilent Mass Hunter Acquisition software version B.02.01 (B2116.30). Ionisation mode: Electrospray lonisation; Drying gas flow: 7 L/min; Nebuliser: 35 psi; Drying gas temperature: 325° C.; Capillary Voltage (Vcap): 4000 V; Fragmentor: 300 V; Skimmer: 65 V; OCT RFV: 250 V; Scan range acquired: 300-3200 m/z Internal Reference ions: Positive Ion Mode=m/z=121.050873 & 922.009798. Protein desalting and chromatographic separation was performed using an Agilent Poroshell C₁₈ 2.1×75 mm, 5 μm column using 5% (v/v) acetonitrile ported to waste (0-5 min). Upon desalting of the sample, the flow was ported back into the ESI source for subsequent gradient elution with (5% (v/v) to 100% (v/v)) acetonitrile/0.1% formic acid over 8 min at 0.25 mL/min. Analysis was performed using Mass Hunter version B.06.00 with BioConfirm software using the maximum entropy protein deconvolution algorithm; mass step 1 Da; Baseline factor 3.00; peak width set to uncertainty.

Non-radioactive analytical HPLC were performed on Agilent 1200 series HPLC system fitted with an Alltech Hypersil BDS-C18 (4.6×150 nm, 5 μm, column A) or a Phenomonex Luna C18(2) column (4.6 mm×150 mm, 5 μm, column B) and Phenomenex SecurityGuard™ C18 guard cartridge (4 mm×30 mm) with a 1 mL/min flow rate; system A: gradient elution of Buffer A=0.1% TFA in H₂O and Buffer B=0.1% TFA in acetonitrile (0 to 100% B in A over 25 min) and UV detection at λ 220, 254, 280 nm and 350 nm.

Several chromatographic systems were used for purification steps. Semipreparative RP—HPLC (Agilent 1200 series HPLC system on a Lunar C18 column, 100 Å 21.2×250 mm, 5 μm) with an 8 mL/min flow rate. System B: gradient elution of Buffer A=0.1% TFA in H₂O and Buffer B=0.1% TFA in acetonitrile (0 to 40% B in A at 30 min, 40-100% B in A at 35 min, 100% B at 40 min) and detection at 214 and 254 nm. System C: System B with the following gradient (0 to 100% B in A at 40 min, 100% B at 44 min, 100 to 0% B in A at 45 min).

Radioactivity was measured using either a Capintec CRC-55tPET dose calibrator set to cal #108 or a PerkinElmer Wizard 2-2470 automatic gamma counter set to 320-500 keV energy window (Bi-213).

Protein concentration was determined using a Thermo Scientific NanoDrop Lite spectrophotometer with blank readings subtracted for the respective vehicle buffer before measurements.

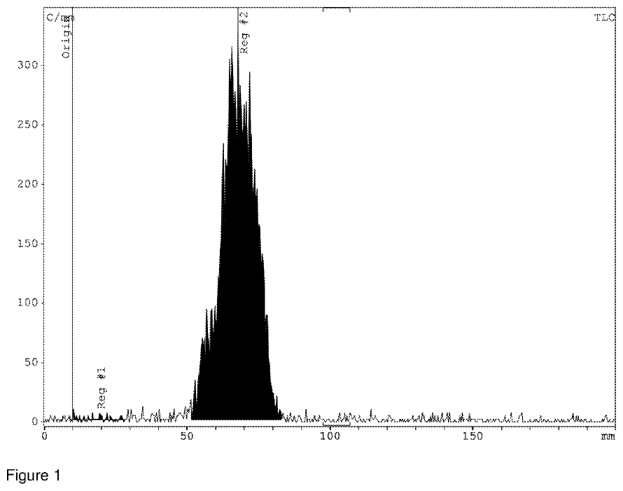

Thin-layer chromatography (TLC) was performed using aluminium-backed silica gel 60 F₂₅₄ strips (Merck, Darmstadt, Germany) and 0.4 M sodium citrate pH 4+10% methanol as a mobile phase. Developed TLC strips were measured at secular equilibrium (min. 8 hours after development) using an Elysia-Raytest Gina Star TLC reader. The chromatogram of the control (i.e. no chelator) is shown in FIG. 1. This shows that, as expected, the actinium-containing solution moves with the solvent front (R_{f}=1) when H₂macropa or H₂macropa-tzPEG₃SqOEt are not present.

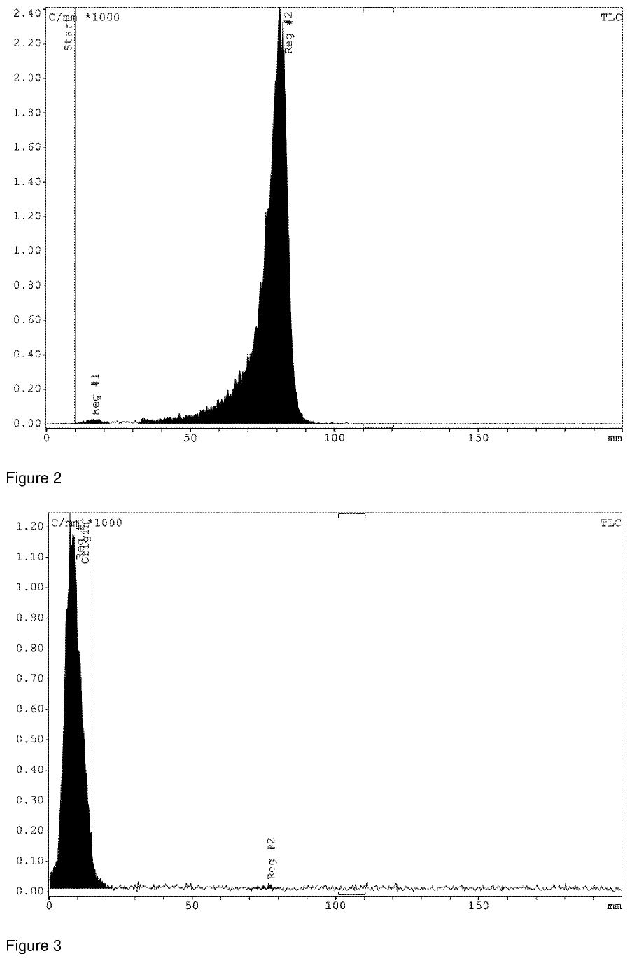

Instant TLC (iTLC) was performed using glass microfibre iTLC-SG chromatography paper strips (Agilent, CA, USA) and either 50 mM EDTA pH 5 or 50 mM citric acid pH 5 as mobile phase. Developed iTLC strips were measured at secular equilibrium (min. 8 hours after development) using an Elysia-Raytest Gina Star TLC reader. The chromatogram of the control (i.e. no chelator) is shown in FIG. 2. This shows that, as expected, the actinium-containing solution moves with the solvent front (R_{f}=1) when H₂macropa-tzPEG₃Sq-conjugated monoclonal antibody is not present.

Size-exclusion HPLC (SE-HPLC) was performed on a 1200 Series Agilent system equipped with a fraction collector and diode-array detector using a Phenomenex BioSep-SEC-S3000 5 μm 300×7.8 mm column and a mobile phase consisting of 50 mM phosphate buffer pH 7.2, 0.2 M NaCl, 5% isopropanol, and 0.02% NaN₃ at a flow rate of 1 mL/min.

Immunoreactive fraction was determined by incubating radioimmunoconjugates (20 ng) in relevant cell lines (5×10⁶ cells) for 45 minutes at ambient temperature followed by spinning the cell suspension (2000 rcf for 2 min) and washing the resulting cell pellet with media (1 mL). Washing was repeated further two times and the activity in the final cell pellet was measured at secular equilibrium (min. 8 hours post wash) using an automated gamma counter. Immunoreactivity or immunoreactive fraction (IRF) was calculated as the fraction of activity in the cell pellet compared to standards with radioimmunoconjugate (20 ng, 500 μL, average of triplicate). Non-specific binding (NSB) was determined by incubating radioimmunoconjugates (20 ng) together with the respective unconjugated monoclonal antibody (60 μg) following the procedure above.

Ac-225 produced from thorium-229 was purchased from Oak Ridge National Laboratory (USA) as a nitrate. [²²⁵Ac]Ac-nitrate (approx. 25-30 MBq) was reconstituted in hydrochloric acid (100 μL, 0.2 M) which was prepared by diluting 30% Suprapur HCl (Sigma) with Ultrapur water (Sigma). Stock solutions of reconstituted [²²⁵Ac]Ac-nitrate with similar activity concentration were prepared by dilution in 0.15 M sodium acetate pH 5.5 (1:9-1:1 depending on Ac-225 decay).

Purified mouse-human IgG1 chimeric monoclonal antibodies EGFRVIII IgG1, EphA3 IgG1, and chimeric IgG1 isotype control, and humanised IgG1 isotype control antibody were provided by the Olivia Newton-John Cancer Research Institute.

U251 glioblastoma cell line was obtained from the American Type Culture Collection (ATCC) and cultured in RPMI media containing 10% fetal calf serum (FCS). The U87MG.de2-7 glioblastoma cell line was provided by the Ludwig Institute for Cancer Research and has been described previously (Nishikawa R, Ji X D, Harmon R C, C S Lazar C S, Gill G N, Cavenee W K, Huang H J. A mutant epidermal growth factor receptor common in human glioma confers enhanced tumorigenicity. Proc Natl Acad Sci USA 1994; 91:7727-31). Cells were cultured in DMEM media containing 10% FCS and 0.4 mg/mL geneticin. SK—RC-52 renal cell carcinoma cell line was provided by Catholic University of Nijmegen (The Netherlands) and cultured in RPMI medium with 10% FCS, 2 mM GlutaMAX (Gibco), and 100 units/mL of penicillin and 100 μg/mL of streptomycin. All cultures were incubated at 37° C. with 5% CO₂.

The majority of the materials were purchased commercially as reagent grade from readily available source.

### Example 1—Dimethylpyridine-2,6-dicarboxylate (100)

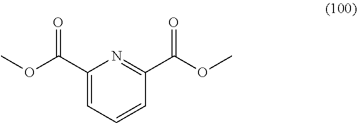

100 was prepared according to an adaption of a literature procedure (Tetrahedron 2015, 71 (33), 5321-5336). Pyridine-2,6-dicarboxylic acid (4.23 g, 25.3 mmol) and sulfuric acid (1 mL) in methanol (30 mL) were heated at reflux for 6 h. The solid was filtered as colourless crystalline solid. A second crop was obtained by removing the solvent under reduced pressure, dissolved in dichloromethane (100 mL) and washed with saturated NaHCO₃ solution (2×50 mL) and water (50 mL). The combined organic layers were dried over MgSO₄ and the solvent was removed under reduced pressure to give colourless solid (3.94 g, 80%). ESI-MS {M+H⁺}: 196.0599 calculated for (C₉H₁₀NO₄)⁺: 196.0605. ¹H NMR (400 MHz, CDCl₃) δ 8.30 (d, J=7.8 Hz, 2H), 8.01 (t, J=7.8 Hz, 1H), 4.01 (s, 6H). ¹³C NMR (101 MHz, CDCl₃) δ 165.0, 148.2, 138.4, 128.0, 53.2. Rt=9.70 min (method A, column B).

### Example 2—Methyl 6-(hydroxymethyl)picolinate (101)

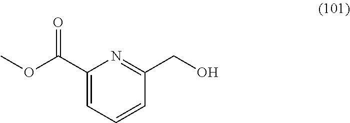

Methyl 6-(hydroxymethyl)picolinate was synthesized using an adapted literature protocol. Inorg. Chem. 2008, 47 (17), 7840-51. To dimethyl 2,6-pyridine-2,6-dicarboxylate (6.0 g, 30.6 mmol) in methanol (200 mL) at 0° C. was added sodium borohydride (2.32 g, 62 mmol) over 1 hour. The reaction mixture was stirred for 5 h at room temperature. The solution was quenched using saturated NH₄Cl (100 mL) at 0° C. and methanol was removed under reduced pressure. The aqueous layer was extracted with dichloromethane (3×100 mL). The combined organic layers were dried over MgSO₄, filtered and the solvent was removed under reduced pressure. The colourless solid was purified by column chromatography (0-2% MeOH in DCM) to give compound 101 (3.37 g, 57%). ESI-MS {M+H⁺}: 168.0652 calculated for (C₃H₁₀NO₃)⁺: 168.0655. ¹H NMR (400 MHz, CDCl₃): δ 8.01 (d, J=7.7 Hz, 1H), 7.83 (t, J=7.7 Hz, 1H), 7.53 (d, J=7.8 Hz, 1H), 4.85 (s, 2H), 3.97 (d, J=0.7 Hz, 3H). ¹³C NMR (125.7 MHz, CDCl₃): δ 165.0, 148.2, 138.4, 128.0, 53.2. Rt=6.67 min (method A, column A).

### Example 3—Methyl-6-chloromethyl-pyridine-2-carboxylate (102)

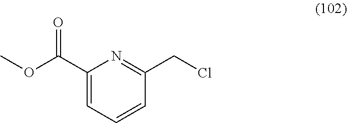

Methyl-6-chloromethyl-pyridine-2-carboxylate was synthesized using an adapted literature protocol.² Thionyl Chloride (6 mL) was slowly added to methyl-6-hydroxymethyl-2-pyridine carboxylate (2.5 g, 15 mmol) at 0° C. under an atmosphere of N₂ and stirred for 1 hour. After 1 hour, the thionyl chloride was removed in vacuo. The residue was dissolved in toluene (50 mL) and washed with saturated NaHCO₃ (50 mL). The organic fractions were dried over MgSO₄, filtered and the solvent was removed under reduced pressure to give an oil which precipitated out to yield an off white solid (2.42 g, 90%). ESI-MS {M+H⁺}: 186.0319 calculated for (CH₉ClNO₂)⁺: 186.0244. ¹H NMR (500 MHz, CDCl₃) δ 8.09 (d, J=7.7 Hz, 1H), 7.91 (t, J=7.8 Hz, 1H), 7.74 (d, J=7.8 Hz, 1H), 4.79 (s, 2H), 4.02 (s, 3H). ¹³C NMR (101 MHz, CDCl₃) δ 165.3, 157.2, 147.5, 138.1, 126.2, 124.5, 53.1, 46.3. Rt=7.65 mins (method A, column A).

### Example 4—Diethyl-4-hydroxypyridine-2,6-carboxylate (103)

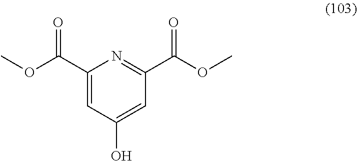

Diethyl-4-hydroxypyridine-2,6-carboxylate was synthesized using an adapted literature protocol.³ Chelidamic acid (4.0 g, 21.8 mmol) was dissolved in ethanol (150 mL) and sulfuric acid (6 drops) was added and refluxed for 16 hours. The ethanol was removed under reduced pressure and used in the next reaction without further purification (3.98 g, 76%). ESI-MS {M+H⁺}: 240.0866 calculated for (C₁₁H₁₄NO₅)⁺: 240.0867. 124° C. ¹H NMR (400 MHz, CDCl₃): δ 9.40 (br, 1H), 7.45 (s, 2H), 4.45 (q, 4H), 1.41 (t, 6H). Rt=7.85 mins (method A, column A).

### Example 5—Diethyl 4-(prop-2-yn-1-yloxy)pyridine-6-dicarboxylate (104)

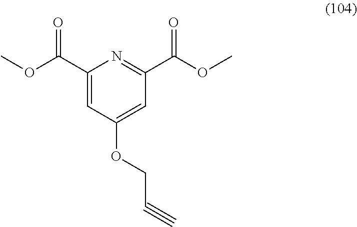

Diethyl 4-(prop-2-yn-1-yloxy)pyridine-2,6-dicarboxylate was synthesized using an adapted literature protocol.⁴ To a suspension of diethyl chelidamate (4) (3.15 g, 13.2 mmol) and K₂CO₃ (3.6 g, 26.4 mmol) in DMF (30 ml) was added propargyl bromide solution (80 wt. % in toluene—4.69 mL, 52.3 mmol). The mixture was heated at 80° C. for 3 h to aid solubility then stirred at room temperature overnight. Then the mixture was filtered, and the filtrate was concentrated under reduced pressure. The residue was purified by column chromatography (0-2% MeOH in DCM) to give compound 104 as either pale yellow or brown solid (2.47 g, 68%). ESI-MS {M+H⁺}: 278.1021 calculated for (C₁₄H₁₆NO₅)⁺: 278.1023 ¹H NMR (400 MHz, DMSO-d₆) δ 7.80 (s, 2H), 5.11 (s, 2H), 4.38 (q, J=7.1 Hz, 4H), 3.75 (s, 1H), 1.34 (t, J=7.1 Hz, 6H). ¹³C NMR (101 MHz, DMSO-d₆) δ 165.5, 164.4, 150.1, 114.9, 80.3, 78.1, 62.1, 57.0, 39.7, 14.5. Rt=8.75 mins (method A, column A).

### Example 6—2-Hydroxymethyl 4-(prop-2-yn-1-yloxy)pyridine-6-ethylcarboxylate (105)

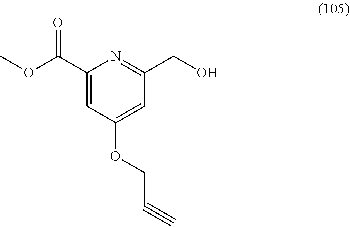 [SMILES: C#CCOc1cc(CO)nc(C(=O)OC)c1]

2-Hydroxymethyl 4-(prop-2-yn-1-yloxy)pyridine-6-ethylcarboxylate was synthesized using an adapted literature protocol.² To Diethyl 4-(prop-2-yn-1-yloxy)pyridine-2,6-dicarboxylate (1.81 g, 6.49 mmol) in ethanol (100 mL) at 0° C. was added sodium borohydride (0.319 g, 8.44 mmol) over 0.5 hours. The reaction mixture was stirred for 3 h at room temperature and monitored via MS until no starting material remained. The solution was quenched using saturated NH₄Cl (50 mL) at 0° C. and ethanol was removed under reduced pressure. The aqueous layer was extracted with ethyl acetate (3×70 mL). The combined organic layers were dried over MgSO₄, filtered and the solvent was removed under reduced pressure to give the off-white compound 6 without further purification (1.2 g, 75%). ESI-MS {M+H⁺}: 236.0919 calculated for (C₁₂H₁₄NO₄)⁺: 236.0918. ¹H NMR (500 MHz, Chloroform-d) δ 7.62 (s, 1H), 7.09 (s, 1H), 4.81 (s, 4H), 4.45 (q, J=7.1 Hz, 2H), 2.60 (t, J=2.3 Hz, 1H), 1.42 (t, J=7.1 Hz, 3H). ¹³C NMR (126 MHz, Chloroform-d) δ 165.2, 164.7, 162.1, 148.8, 111.4, 109.7, 76.7, 64.5, 62.1, 56.0, 14.3. Rt=11.32 mins (method A, column A).

### Example 7—2-Chloromethyl 4-(prop-2-yn-1-yloxy)pyridine-6-ethylcarboxylate (106)

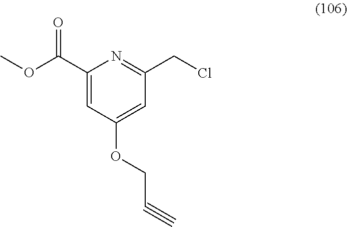 [SMILES: C#CCOc1cc(CCl)nc(C(=O)OC)c1]

Thionyl Chloride (6 mL) was slowly added to 2-Hydroxymethyl 4-(prop-2-yn-1-yloxy)pyridine-6-ethylcarboxylate (0.4 g, 1.7 mmol) at 0° C. under an atmosphere of N₂ and stirred for 3 hours. After 3 hours, the thionyl chloride was removed in vacuo to yield a pale yellow residue, which was dissolved in ethyl acetate (20 mL) and washed with saturated NaHCO₃ (30 mL) and water (30 mL). The organic fractions were dried over MgSO₄, filtered and the solvent was removed under reduced pressure to yield a pale-yellow powder (0.33 g, 77%). ESI-MS {M+H⁺}: 254.0578 calculated for (C₁₂H₁₄NO₄)⁺: 254.0578. ¹H NMR (500 MHz, Chloroform-d) δ 7.62 (s, 1H), 7.09 (s, 1H), 4.81 (s, 4H), 4.45 (q, J=7.1 Hz, 2H), 2.60 (t, J=2.3 Hz, 1H), 1.42 (t, J=7.1 Hz, 3H). ¹³C NMR (126 MHz, Chloroform-d) δ 165.2, 164.7, 162.1, 148.8, 111.4, 109.7, 76.7, 64.5, 62.1, 56.0, 14.3. Rt=14.942 mins (method A, column B).

### Example 8—Methyl 6-((1,4,10,13-tetraoxa-7,16-diazacyclooctadecan-7-yl)methyl)picolinate (107)

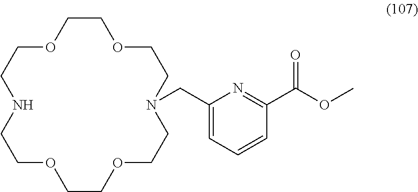 [SMILES: COC(=O)c1cccc(CN2CCOCCOCCNCCOCCOCC2)n1]

Methyl 6-((1,4,10,13-tetraoxa-7,16-diazacyclooctadecan-7-yl)methyl)picolinate was synthesized using an adapted literature protocol.⁵ To a solution of 1,7,10,16-tetraoxa-4,13-diazacyclooctadecane (0.77 g, 3.02 mmol) and DIPEA (0.34 g, 3.93 mmol) in dry ACN (350 mL) at 75° C. was added dropwise a solution of 102 (0.37 g, 2.03 mmol) in dry ACN (80 mL) over 4 hours under an atmosphere of nitrogen and was heated at reflux for 40 h. The solution was concentrated at 60° C. under reduced pressure to a pale-yellow oil. To the crude oil was then purified by via semi-preparative HPLC (method C) and appropriate fractions were combined and lyophilised to give a pale oil (626 mg, 76%). ESI-MS {M+2H⁺}: 206.6257 calculated for (C₂₀H₃₃N₃O₆)²⁺: 206.6257. ¹H NMR (500 MHz, Chloroform-d) δ 9.57 (s, 1H), 8.07 (d, J=7.7 Hz, 1H), 7.91 (t, J=7.7 Hz, 1H), 7.85 (d, J=7.7 Hz, 1H), 4.97 (s, 2H), 3.97 (s, 3H), 3.89 (s, 4H), 3.82 (s, 4H), 3.75 (s, 3H), 3.61 (s, 9H), 3.33 (s, 4H). ¹³C NMR (126 MHz, cdcl₃) δ 165.0, 161.6-160.7 (TFA, q), 152.0, 146.9, 138.5, 128.5, 124.6, 119.6-112.6 (TFA, q), 70.1, 69.4, 65.8, 65.5, 54.5, 52.9, 52.5, 48.6. Rt=7.26 min (method A, column A).

### Example 9—Ethyl 4-(prop-2-yn-1-yloxy)-6-((16-((6-(methoxycarbonyl)pyridin-2-yl)methyl)-1,4,10,13-tetraoxa-7,16-diazacyclooctadecan-7-yl)methyl)picolinate (108)

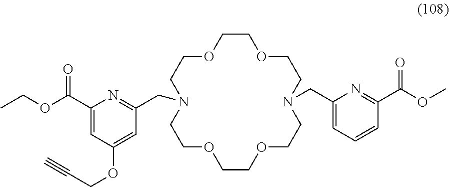 [SMILES: C#CCOc1cc(CN2CCOCCOCCN(Cc3cccc(C(=O)OC)n3)CCOCCOCC2)nc(C(=O)OCC)c1]

Caesium Carbonate (0.65 g, 2.0 mmol) was added to a solution of methyl 6-((1,4,10,13-tetraoxa-7,16-diazacyclooctadecan-7-yl)methyl)picolinate (0.149 g, 0.4 mmol) in anhydrous DMF (10 mL) under an atmosphere of nitrogen and stirred at room temperature for 30 minutes. To the stirred reaction mixture was added a solution of 2-chloromethyl 4-(prop-2-yn-1-yloxy)pyridine-6-ethylcarboxylate (0.138 g, 0.54 mmol) in anhydrous DMF (2 mL) under nitrogen and the reaction was heated at 50° C. for 40 hours. The resulting solution was filtered and taken to dryness under reduced pressure to yield an orange oil, which was purified via semi preparative HPLC and appropriate fractions were combined and lyophilised to give a yellow oil. ESI-MS {M+2H⁺}: 315.1627 calculated for (C₃₂H₄₆N₄O₉)²⁺: 315.1627. ¹H NMR (500 MHz, CDCl₃) δ 8.11 (d, J=7.2 Hz, 1H), 7.94 (t, J=7.6 Hz, 1H), 7.80 (d, J=6.7 Hz, 1H), 7.69 (s, 1H), 7.44 (s, 1H), 4.84 (s, 2H), 4.75 (s, 2H), 4.69 (s, 2H), 4.47-4.39 (m, 2H), 3.98 (d, J=2.7 Hz, 3H), 3.95 (s, 8H), 3.65 (d, J=3.0 Hz, 16H), 2.61 (s, 1H), 1.40 (t, J=8.3 Hz, 3H). Rt=10.23 mins (method A, column A).

### Example 10—4-(prop-2-yn-1-yloxy)-6-((16-((6-carboxypyridin-2-yl)methyl)-1,4,10,13-tetraoxa-7,16-diazacyclooctadecan-7-yl)methyl)picolinic acid (109)

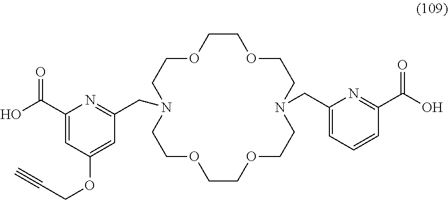 [SMILES: C#CCOc1cc(CN2CCOCCOCCN(Cc3cccc(C(=O)O)n3)CCOCCOCC2)nc(C(=O)O)c1]

4-(prop-2-yn-1-yloxy)-6-((16-((6-carboxypyridin-2-yl)methyl)-1,4,10,13-tetraoxa-7,16-diazacyclooctadecan-7-yl)methyl)picolinic acid was synthesized using an adapted literature protocol.⁶ To a solution of 108 (18.5 mg, 0.03 mmol) in DCM (450 μL), was added a sonicated suspension of NaOH in methanol (50 μL, 3 M), so the final concentration of NaOH was 0.3 M and the final DCM:MeOH ratio was 9:1. The reaction mixture was stirred at room temperature for 1 hour, then the solvent was removed under reduced pressure and used in the next step without further purification. ESI-MS {M+2H⁺}: 294.1393 calculated for (C₂₉H₄₀N₄O₉)²⁺: 294.1393. Rt=8.53 mins (method A, column A).

### Example 11—N₃-PEG₃-Squarate (110)

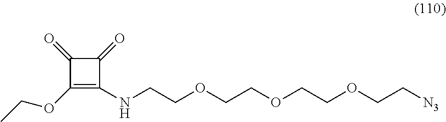 [SMILES: CCOc1c(NCCOCCOCCOCCN=[N+]=[N-])c(=O)c1=O]

Freshly prepared diethyl squarate (0.23 g, 1.32 mmol) was dissolved in ethanol (4 mL) before adding 1-Amino-11-azido-3,6,9-trioxaundecane (0.28 g, 1.29 mmol) and DIPEA (1 eq) under nitrogen. The mixture was stirred at room temperature for 8 hours then the colourless solution was taken to dryness under reduced pressure and purified via semi preparative HPLC (method C). Fractions were combined and lyophilized to yield a colourless oil (209 mg, 49%). ESI-MS {M+H⁺}: 343.1613 calculated for (C₁₄H₂₃N₄O₆)⁺: 343.1612 ¹H NMR (500 MHz, Chloroform-d) δ 4.75 (d, J=6.7 Hz, 2H), 3.66 (d, J=8.9 Hz, 14H), 3.42-3.36 (m, 2H), 1.45 (t, J=7.1 Hz, 3H). 13-C NMR (126 MHz; cdcl₃): δ 188.8, 183.3, 177.4, 177.2, 172.7, 70.7, 70.3, 70.0, 69.73, 69.61, 50.7, 44.4, 44.1, 15.8. RT=7.23 mins (method A, column A).

### Example 12—CuAAC Between N₃-PEG₃-Squarate and 4-(prop-2-yn-1-yloxy)-6-((16-((6-carboxypyridin-2-yl)methyl)-1,4,10,13-tetraoxa-7,16-diazacyclooctadecan-7-yl)methyl)picolinic acid (111)

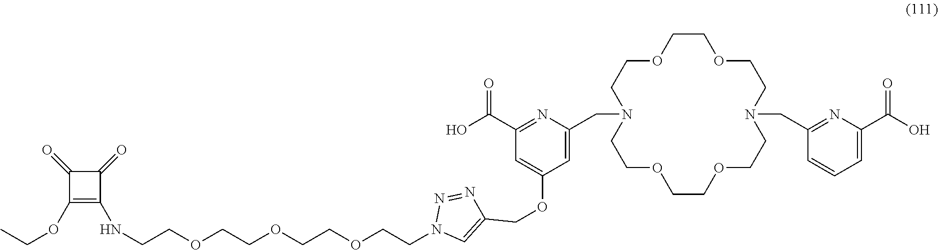 [SMILES: CCOc1c(NCCOCCOCCOCCn2cc(COc3cc(CN4CCOCCOCCN(Cc5cccc(C(=O)O)n5)CCOCCOCC4)nc(C(=O)O)c3)nn2)c(=O)c1=O]

CuSO₄ (0.22 mg, 0.0014 mmol, 8% mmol) and Sodium Ascorbate (0.64 mg, 0.0032 mmol, 18% mmol) were combined and shaken for 2 minutes to ensure complete reduction to copper(I) species. TBTA (2.8 mg, 0.0057 mmol, 32% mmol in DMF) was then added as a stabilizing ligand with N₃-PEG₃-Squarate (6.42 mg, 0.018 mmol) and reacted for 10 minutes. 109 (11 mg, 0.018 mmol) was added, and the reaction mixture was purged for 15 minutes with a stream of nitrogen to reduce the oxygen content in the reaction mixture. The reaction was monitored via HPLC (280 nm) and a further 2% mmol CuSO₄ and 18% mmol Sodium Ascorbate were added after 12 hours to ensure reaction had gone to completion. The yellow solution was taken to dryness under reduced pressure and dried in vacuo. The resulting precipitate was purified via semi-preparative HPLC and fractions were combined and lyophilised to yield an off-white powder (5 mg, 40%). ESI-MS {M+2H⁺}: 465.2161 calculated for (C₄₃H₆₂N₈O₁₅)²⁺: 465.2162.

¹H NMR (500 MHz, Chloroform-d) δ 8.11 (d, J=7.2 Hz, 1H), 7.94 (t, J=7.6 Hz, 1H), 7.80 (d, J=6.7 Hz, 1H), 7.69 (s, 1H), 7.44 (s, 1H), 4.84 (d, J=2.8 Hz, 2H), 4.75 (s, 2H), 4.69 (s, 2H), 4.47-4.39 (m, 2H), 3.98 (d, J=2.7 Hz, 3H), 3.95 (s, 8H), 3.72-3.58 (m, 16H), 2.61 (s, 1H), 1.40 (t, J=8.3 Hz, 3H). Rt=10.04 mins (method A, column A).

### Example 13—Protein Conjugations—Herceptin

Lyophilized Herceptin (Trastuzumab—500 mg) was reconstituted in borate buffer (0.2 M, pH 9.0) to a final concentration of 10 mg/mL. Macropa-PEG₃-SqOEt (Compound 111, 6 μL of 5 mg/mL stock solution in DMSO, 10 equivalents) was added and the reaction was incubated in the dark at room temperature for 6 hours before excess reagents were removed and buffer exchanged (HEPES, 0.1 M, pH7.4) via spin filtration (50 KDa MW cut off).

### Example 14—EGFRVIII IgG1 and EphA3 IgG1 Antibody Conjugations

EGFRVIII IgG1 (9.4 mg/mL) and EphA3 IgG1 antibodies (3.1. mg/mL; 3 mg, 2×10⁻⁵ mmol) in PBS were buffer exchanged into borate buffer (0.2 M, pH 9.0) and divided into 2 equal aliquots to a final concentration of 5 mg/mL (total volume 300 μL for EGFRVIII IgG1 and 320 μL for EphA3 IgG1. To the reaction mixture was added H₂macropa-tzPEG₃Sq OEt (111, 10 mg/mL stock solution in DMSO—final DMSO concentration less than 4%) and shaken overnight at 4° C., then at room temperature for a second night. Excess reagents were removed and buffer exchanged (Sodium Acetate, 0.1 M, pH 5.5) via spin filtration (50 KDa MW cut off).

| | EGFRVIII IgG1 | | EphA3 IgG1 | |
|---|---|---|---|---|
| Concentration post buffer exchange | 6.6 | mg/mL | 5.7 | mg/mL |
| Total volume | 300 | μL | 320 | μL |
| Mass used | 1.5 | mg | 1.275 | mg |
| Cmpd 111 added (8:1 equivalents) | 7.5 | μL | 6.4 | μL |
| Cmpd 111 added (15:1 equivalents) | 14 | μL | 12 | μL |

### Example 15—Ac-225 Labelling Concentration Study

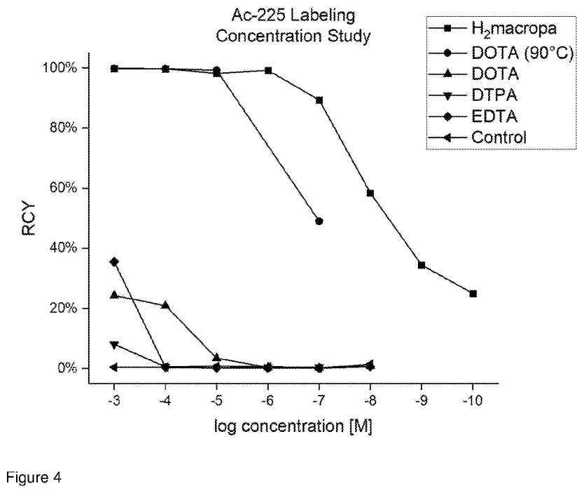

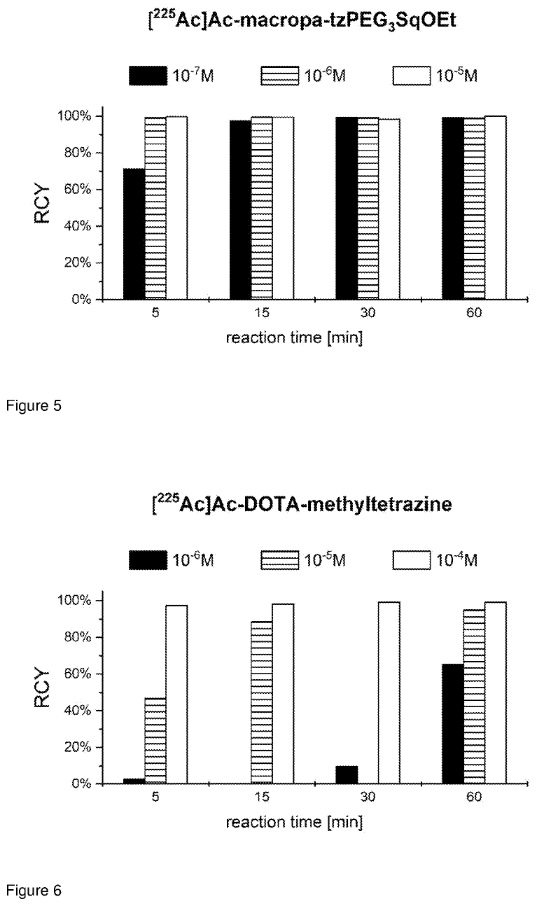

Ac-225 stock solution (approx. 74 kBq, 2.3 μL) was added to solutions of H₂macropa, DOTA (2,2′,2″,2″′-(1,4,7,10-Tetraazacyclododecane-1,4,7,10-tetrayl)tetraacetic acid), DTPA (Diethylenetriamine pentaacetate), EDTA (ethylenediaminetetraacetic acid) and buffer control to give mixtures of 100 μL total volume with chelator concentrations between 10⁻³ M-10⁻¹⁰ M. Reaction mixtures were incubated at ambient temperature for 2 hours unless noted otherwise and samples for TLC analysis (0.4 M sodium citrate pH 4+10% MeOH, product R_{f}=0) were taken at the conclusion of the reaction. An exemplary chromatogram of [²²⁵Ac]Ac-macropa is shown in FIG. 3. This shows that actinium has been retained on the base line (i.e. the “origin”) by virtue of it being complexed to macropa. Radiochemical yields are presented in FIG. 4.

Radiochemical yields of [²²⁵Ac]Ac-macropa are notably higher at ambient temperature compared to the other chelators investigated. H₂macropa can be radiolabelled quantitatively up to a chelator concentration of 10⁻⁶ M. Other chelators such as DOTA, DTPA, and EDTA show no complexation of [²²⁵Ac]Ac at lower chelator concentrations, similar to the control (no chelator). Radiolabelling of DOTA displays quantitative radiolabelling yields when heated to 90° C., although slightly inferior compared to H₂macropa at lower chelator concentrations. These findings are in line with published observations: N. A. Thiele, V. Brown, J. M. Kelly, A. Amor-Coarasa, U. Jermilova, S. N. MacMillan, A. Nikolopoulou, S. Ponnala, C. F. Ramogida, A. K. H. Robertson, C. Rodríguez-Rodríguez, P. Schaffer, C. Williams, J. W. Babich, V. Radchenko, J. J. Wilson, Angew. Chem. Int. Ed. 2017, 56, 14712.

### Example 16 Speed of Complexation of Compound 111 with Ac-225 Comparison Study

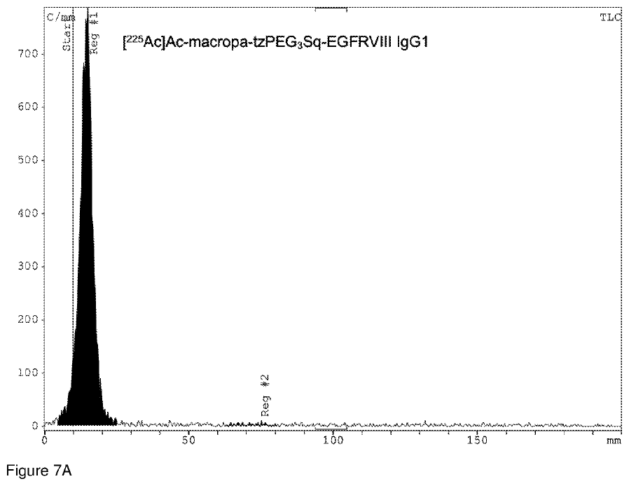

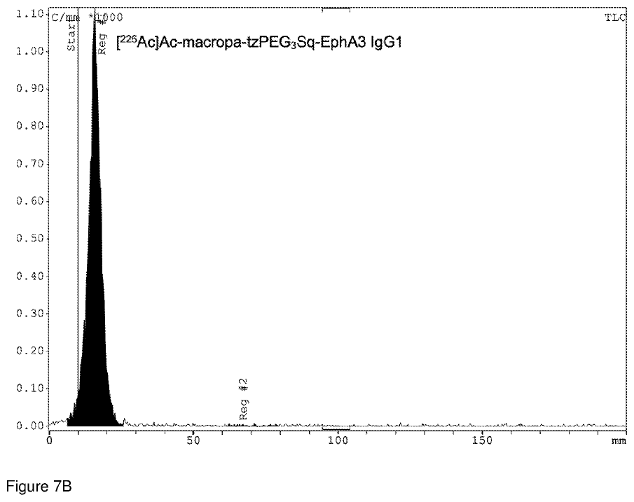

Ac-225 stock solution (approx. 74 kBq, 2.3 μL) was added to solutions of H₂macropa-tzPEG₃SqOEt or DOTA-methyltetrazine to give mixtures of 100 μL total volume with chelator concentrations of 10⁻⁵ M, 10⁻⁶ M, 10⁻⁷ M and 10⁻⁴ M, 10⁻⁵ M, 10⁻⁶ M, respectively. Reaction mixtures of H₂macropa-tzPEG₃SqOEt and DOTA-methyltetrazine were incubated at ambient temperature and 90° C., respectively, and samples for TLC analysis (0.4 M sodium citrate pH 4+10% MeOH, product R_{f}=0) were taken at 5, 15, 30, and 60 minutes. Radiochemical yields are presented in FIG. 5 and FIG. 6.

Radiolabelling of H₂macropa-tzPEG₃SqOEt proceeded with quantitative yields at all concentrations after incubation for 15 minutes. At 10⁻⁷ M chelator concentration, the radiochemical yield started to drop at 5 minutes incubation. Therefore, 10⁻⁷ and 10⁻⁶ M chelator concentrations and 15 minute incubation at ambient temperature were chosen for radiolabelling studies with H₂macropa-tzPEG₃Sq-conjugated monoclonal antibodies.

Radiolabelling of DOTA-methyltetrazine proceeded with quantitative yields at 10⁻⁴ M chelator concentration at all incubation times. At 10⁻⁵ M chelator concentration, the radiochemical yield was 88.5% at 15 minutes incubation and 10⁻⁶ M resulted in a maximum radiochemical yield of 65.3% at 60 minutes incubation. Therefore, 10⁻⁵ and 10⁻⁴ M chelator concentrations were chosen for radiolabelling studies with TCOPEG₄-conjugated monoclonal antibodies (where TCOPEG-=abbreviation for ‘transcyclooctene polyethylene glycol’).

### Example 17—Synthesis of [²²⁵Ac]Ac-macropa-tzPEG₃Sq-EGFRVIII IgG1 and [²²⁵Ac]Ac-macropa-tzPEG₃Sq-EphA3 IgG1

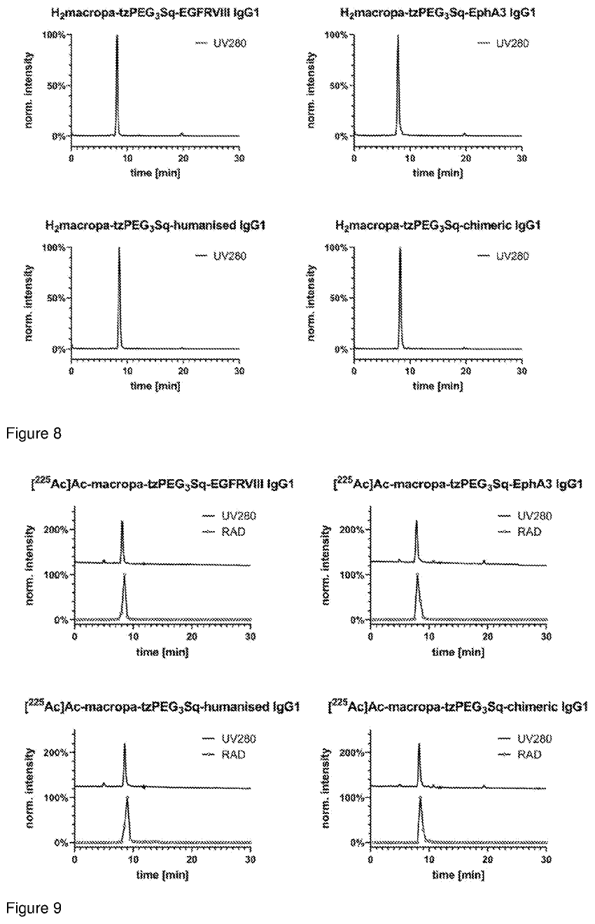

Ac-225 stock solution (555 kBq, 30 μL) was added to solutions of H₂macropa-tzPEG₃Sq-conjugated monoclonal antibodies to give mixtures of 100 μL total volume with conjugate concentrations of 10⁻⁷ M and 10⁻⁶ M. Reaction mixtures were incubated at ambient temperature for 15 minutes and samples were taken for determination of radiochemical purity (RP; iTLC: 50 mM citrate pH 5, product R_{f}=0) and immunoreactivity (IR). Specific activities (SA) were calculated by dividing the starting activity of Ac-225 by the amount of conjugate and multiplying with the radiochemical purity. Results are summarized in Table 1. Numerical indicators (2×, 5×) refer to the average chelator-antibody-ratio of each conjugate. The chromatograms of [²²⁵Ac]Ac-macropa-tzPEG₃Sq-EGFRVIII IgG1 and [²²⁵Ac]Ac-macropa-tzPEG₃Sq-EphA3 IgG1 are shown in FIG. 7A and FIG. 7B, respectively. This shows that actinium has been retained on the base line (i.e. the “origin”) by virtue of it being complexed to macropa-tzPEG₃Sq-EGFRVIII IgG1 or macropa-tzPEG₃Sq-EphA3 IgG1.

Radiolabelling of H₂macropa-tzPEG₃Sq-conjugated monoclonal antibodies with Ac-225 proceeded with radiochemical yields>99.5% at 10⁻⁶ M antibody concentration. At this antibody concentration, specific activities were close to the theoretical maximum of 37 MBq/mg. Radioimmunoconjugates with an average of 2 chelators per antibody resulted in higher immunoreactivity compared to radioimmunoconjugates with an average of 5 chelators per antibody. Therefore, the former were chosen for further studies.

TABLE 1
Radiolabelling and quality control results of [²²⁵Ac]Ac-macropa-tzPEG₃Sq-
EGFRVIII IgG1 and [²²⁵Ac]Ac-macropa-tzPEG₃Sq-EphA3 IgG1 at different antibody
concentrations using immunoconjugates with different chelator-antibody-ratios.

| | Conjugate concentration | | | | | |
|---|---|---|---|---|---|---|
| | 10⁻⁷ M | | | 10⁻⁶ M | | |
| | H₂macropa conc. [M] | | | | | |
| | 2x \| 5x | | | | | |
| | 2 × 10⁻⁷ M \| 5 × 10⁻⁷ M | | | 2 × 10⁻⁶ M \| 5 × 10⁻⁶ M | | |
| | Amount of conjugate | | | | | |
| | 1.5 μg | | | 15 μg | | |
| | Maximum theoretical | | | | | |
| | specific activity (SA) | | | | | |
| | 370 MBq/mg | | | 37.0 MBq/mg | | |
| | RP | IR | SA | RP | IR | SA |
| Analysis | [%] | [%] | [MBq/mg] | [%] | [%] | [MBq/mg] |
| 2x EGFRVIII IgG1 | 1.77 | 1.13 | 6.55 | 99.75 | 83.49 | 36.9 |
| 5x EGFRVIII IgG1 | 98.74 | 66.88 | 365 | 99.81 | 68.22 | 36.9 |
| 2x EphA3 IgG1 | 99.68 | 85.09 | 369 | 99.65 | 101.6 | 36.9 |
| 5x EphA3 IgG1 | 99.85 | 90.22 | 369 | 99.99 | 91.10 | 37.0 |

### Example 18—Size-Exclusion HPLC of Various H₂Macropa-tzPEG₃Sq-Conjugated Monoclonal Antibodies

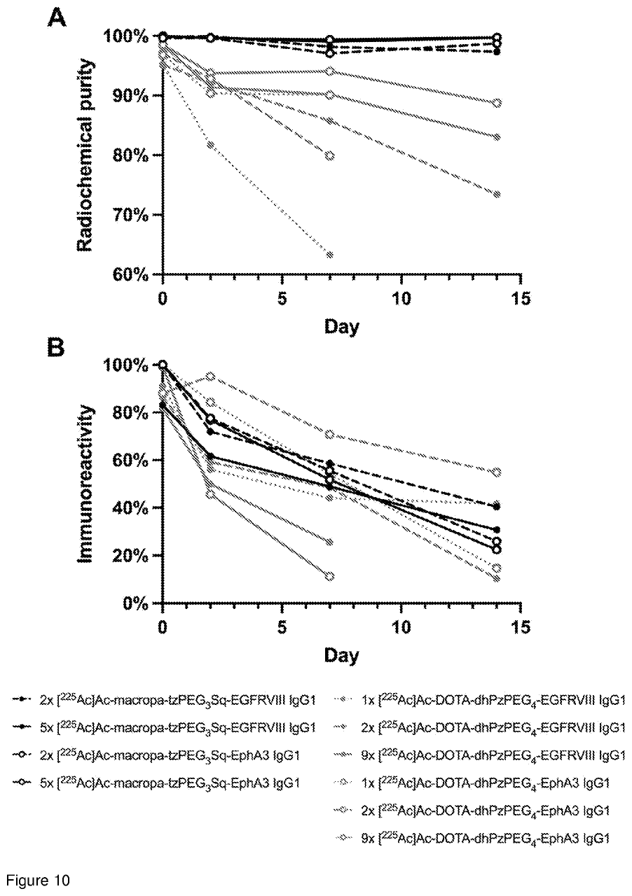

H₂macropa-tzPEG₃Sq-conjugated monoclonal antibodies (20 μg) were analysed using size-exclusion HPLC (SE-HPLC). Retention times are summarized in Table 2. SE-HPLC chromatograms (UV_{280 nm}) are shown in FIG. 8.

SE-HPLC showed excellent antibody integrity for all immunoconjugates with negligible levels of aggregation.

TABLE 2
SE-HPLC retention times of H₂macropa-
tzPEG₃Sq-immunoconjugates (UV₂₈₀).

| | UV₂₈₀ retention |
|---|---|
| Immunoconjugate | time [min] |
| H₂macropa-tzPEG₃Sq-EGFRVIII IgG1 | 8.141 |
| H₂macropa-tzPEG₃Sq-EphA3 IgG1 | 7.867 |
| H₂macropa-tzPEG₃Sq-humanised IgG1 | 8.556 |
| H₂macropa-tzPEG₃Sq-chimeric IgG1 | 8.256 |

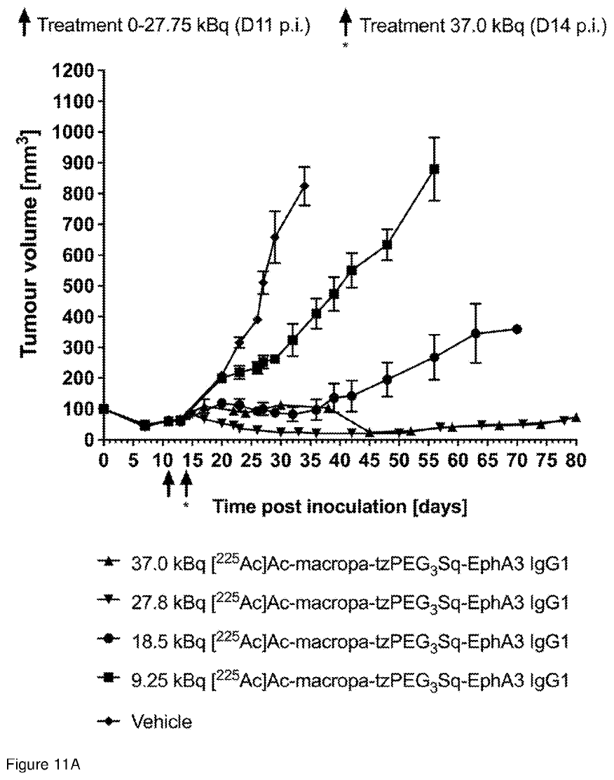

### Example 19—Size-Exclusion HPLC of Various Ac225 Labelled Monoclonal Antibodies

[²²⁵Ac]Ac-macropa-tzPEG₃Sq-conjugated monoclonal antibodies (37 kBq, ˜1 μg) were analysed using SE-HPLC. HPLC fractions were collected continuously every 30 seconds (0.5 mL) into Eppendorf tubes. Radioactivity in the tubes was determined at secular equilibrium (min. 8 hours post collection) using an automated gamma counter. UV_{280 nm} and radiation retention times are summarized in Table 3. SE-HPLC chromatograms (UV₂₈₀ nm and RAD) are shown in FIG. 9.

SE-HPLC showed excellent antibody integrity for all radioimmunoconjugates with negligible levels of aggregation. No free Ac-225 was detected by SE-HPLC which is in agreement with iTLC analyses.

TABLE 3
SE-HPLC retention times of [²²⁵Ac]Ac-macropa-
tzPEG₃Sq-immunoconjugates (UV₂₈₀ and radiation)

| | UV₂₈₀ | RAD* |
|---|---|---|
| | retention | retention |
| Radioimmunoconjugate | time [min] | time [min] |
| [²²⁵Ac]Ac-macropa-tzPEG₃Sq-EGFRVIII IgG1 | 8.119 | 8.5 |
| [²²⁵Ac]Ac-macropa-tzPEG₃Sq-EphA3 IgG1 | 7.865 | 8.0 |
| [²²⁵Ac]Ac-macropa-tzPEG₃Sq-humanised IgG1 | 8.561 | 9.0 |
| [²²⁵Ac]Ac-macropa-tzPEG₃Sq-chimeric IgG1 | 8.310 | 8.5 |

*resolution: 0.5 min; determined via counting of discrete 0.5 mL HPLC fractions at secular equilibrium

### Example 20—Competition Study of [²²⁵Ac]Ac-macropa-tzPEG₃Sq-EGFRVIII IgG1 with La³⁺ and EDTA

[²²⁵Ac]Ac-macropa-tzPEG₃Sq-EGFRVIII IgG1 (37 kBq, 1 μg, 5 μL) was added to mixtures in PBS containing variations of gentisate (0/25 mg/mL), La³⁺ (5/50/500× molar excess relative to antibody), and EDTA (50× molar excess relative to antibody) to give solutions with 20 μL total volume. Mixtures were incubated at ambient temperature and samples for iTLC (50 mM citrate pH 5, product R_{f}=0) analysis were taken at 1, 24, 48, and 168 hours. Radiochemical purities are presented in Table 4.

[²²⁵Ac]Ac-macropa-tzPEG₃Sq-EGFRVIII IgG1 showed excellent stability in PBS with and without sodium gentisate and in competition with La³⁺ up to 50-fold molar excess. At 500-fold molar excess of La³⁺ and 50-fold molar excess of EDTA, the radiochemical purity dropped below 95% between 24-48 hours.

TABLE 4
Stability and competition assay results of
[²²⁵Ac]Ac-macropa-tzPEG₃Sq-EGFRVIII IgG1 at
ambient temperature. Radiochemical purities
determined via iTLC analyses using
50 mM citric acid pH 5.

| | Radiochemical purity [%] @ t0 | | | |
|---|---|---|---|---|
| | 99.66 | | | |
| Radiochemical purity [%] @ time | 1 h | 24 h | 48 h | 168 h |
| in PBS | 98.98 | 100 | 97.11 | 96.85 |
| in PBS + gentisate (25 mg/mL) | 100 | 98.61 | 96.35 | 100 |
| in PBS + gentisate + La³⁺ (5x) | 99.31 | 99.11 | 98.07 | 96.76 |
| in PBS + gentisate + La³⁺ (50x) | 98.99 | 98.95 | 98.78 | 100 |
| in PBS + gentisate + La³⁺ (500x) | 93.16 | 98.31 | 78.44 | 68.89 |
| in PBS + gentisate + EDTA (50x) | 99.27 | — | 84.78 | 65.88 |

### Example 21—Serum Stability Analysis and Comparison to [²²′Ac]Ac-DOTA-dhPzPEG₄-Conjugated Monoclonal Antibodies

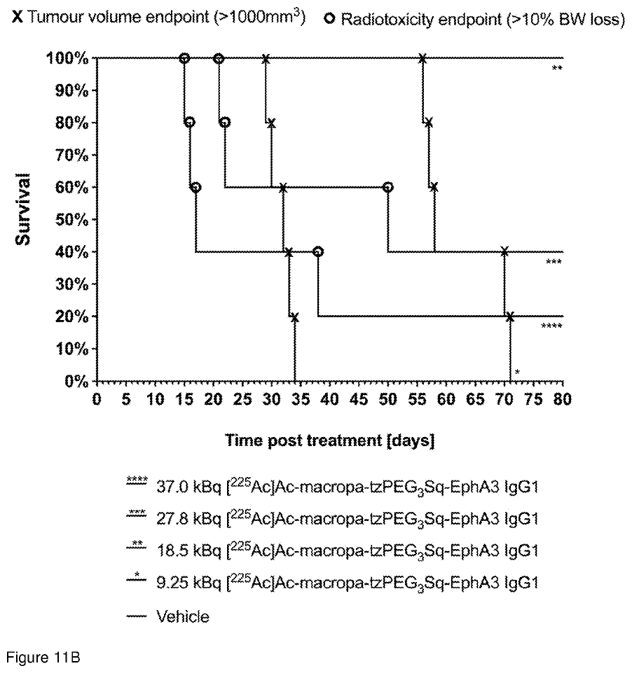

TCOPEG₄-conjugated monoclonal antibodies were prepared similar to published procedures by O. Keinanen, K. Fung, J. Pourat, V. Jallinoja, D. Vivier, N. K. Pillarsetty, A. J. Airaksinen, J. S. Lewis, B. M. Zeglis, M. Sarparanta, EJNMMI Res 2017, 7, 95 and Z. Zhou, N. Devoogdt, M. R. Zalutsky, G. Vaidyanathan, Bioconjug Chem. 2018, 29, 4090-4103. Briefly, monoclonal antibody in sodium bicarbonate (0.1 M, pH 8.5) was incubated with TCO-PEG₄-NHS (4×/10×/40× molar equivalents) for 1 hour at 37° C. followed by purification via centrifugal filtration (50 kDa MWCO) using formulation buffer consisting of sodium acetate (50 mM, pH 5.6), sorbitol (5% w/v), and Tween 20 (0.02% w/v). [²²⁵Ac]Ac-DOTA-dhPzPEG₄-EGFRVIII IgG1 and [²²⁵Ac]Ac-DOTA-dhPzPEG₄-EphA3 IgG1 (where —dhPzPEG—=abbreviation for ‘methyldihydropyridazine polyethylene glycol’) were synthesized by incubating TCOPEG₄-EGFRVIII IgG1 (150 μg) and TCOPEG₄-EphA3 IgG1 (150 μg) with [²²⁵Ac]Ac-DOTA-methyltetrazine (555 kBq) in sodium acetate (0.15 M, pH 5.5) for 15 minutes at ambient temperature followed by purification via spin filtration (10 kDa MWCO). [²²⁵Ac]Ac-macropa-tzPEG₃Sq- and [²²⁵Ac]Ac-DOTA-dhPzPEG₄-conjugated monoclonal antibodies were incubated in healthy donor human serum (100 μL) at 37° C. Samples were taken at EOS and after 2, 7, and 14 days incubation. Samples were analysed by iTLC (50 mM EDTA pH 5, product R_{f}=0) and immunoreactive fraction was determined with U87MG.de2-7 and U251 cells for EGFRVIII IgG1 and EphA3 IgG1 radioimmunoconjugates, respectively. Results are presented in FIG. 10A for radiochemical purity and FIG. 10B for immunoreactivity. Numerical indicators (2×, 5×, 1×, 2×, 9×) refer to the average chelator-antibody-ratio of each conjugate.

Radiochemical purity of [²²⁵Ac]Ac-macropa-tzPEG₃Sq-EGFRVIII IgG1 and [²²⁵Ac]Ac-macropa-tzPEG₃Sq-EphA3 IgG1 in human serum was >95% at the conclusion of the experiment (14 days). [²²⁵Ac]Ac-DOTA-dhPzPEG₄-conjugated monoclonal antibodies collectively showed inferior stability over the investigated timeframe. Apart from [²²⁵Ac]Ac-DOTA-dhPzPEG₄-conjugates with an average chelator-antibody-ratio of 9 which showed inferior characteristics, no distinction could be made between other radioimmunoconjugates on the basis of immunoreactivity. [²²⁵Ac]Ac-macropa-tzPEG₃Sq-conjugates with an average chelator-antibody-ratio of 2 were used for in vivo studies described in Example 23 and Example 24.

### Example 22—Serum Stability Analysis

[²²⁵Ac]Ac-macropa-tzPEG₃Sq-conjugated monoclonal antibodies with an average chelator-antibody-ratio of 2 were incubated in healthy donor human serum (100 μL) at 37° C. Samples were taken at EOS and after 2, 7, and 14 days incubation. Samples were analysed by iTLC (50 mM EDTA pH 5, product R_{f}=0) and immunoreactive fraction was determined with U87MG.de2-7 (EGFRVIII IgG1), U251 (EphA3 IgG1), or SK—RC-52 (chimeric isotype control IgG1) cells. Results are presented in Table 5.

Radiochemical yields and specific activities are very consistently high for all three radioimmunoconjugates and radiochemical purity was excellent over the investigated timeframe (>99% after 14 days). Immunoreactive fraction dropped gradually at each timepoint for all radioimmunoconjugates. Non-specific binding was low in all instances.

TABLE 5
Radiolabelling results and serum stability analyses of [²²⁵Ac]Ac-macropa-
tzPEG₃Sq-conjugated monoclonal antibodies. Radiochemical purities determined
via iTLC analyses using 50 mM citric acid pH 5. Immunoreactive fraction (IRF)
and non-specific binding (NSB) were determined with U87MG.de2-7 (EGFRVIII IgG1),
U251 (EphA3 IgG1), or SK-RC-52 (chimeric IgG1 isotype control) cells.

| | | | chimeric IgG1 |
|---|---|---|---|
| Radioimmunoconjugate | EGFRVIII IgG1 | EphA3 IgG1 | Isotype control |
| RCY = RP @ D 0 [%] | 99.68 | 99.83 | 99.68 |
| SA [MBq/mg] | 36.9 | 36.9 | 36.9 |
| IRF \| NSB | 71.76 \| 2.45 | 100 \| 0.13 | 89.17 \| 5.54 |

Serum Stability

| | | | |
|---|---|---|---|
| RP (D 2 \| D 7 \| D 14) | 99.52 \| 99.32 \| 99.94 | 99.56 \| 99.91 \| 99.51 | 99.47 \| 99.76 \| 99.72 |
| IRF (D 2 \| D 7 \| D 14) | 61.13 \| 60.00 \| 42.84 | 76.04 \| 59.76 \| 33.59 | 82.35 \| 68.61 \| 52.84 |
| NSB (D 2 \| D 7 \| D 14) | 0.75 \| 0.65 \| 0 | 0 \| 0 \| 0 | 2.62 \| 5.85 \| 0.78 |

### Example 23—Dose Escalation Studies of [²²⁵Ac]Ac-Macropa-tzPEG₃Sq-EphA3 IgG1 in Mice Bearing U251 Xenografts

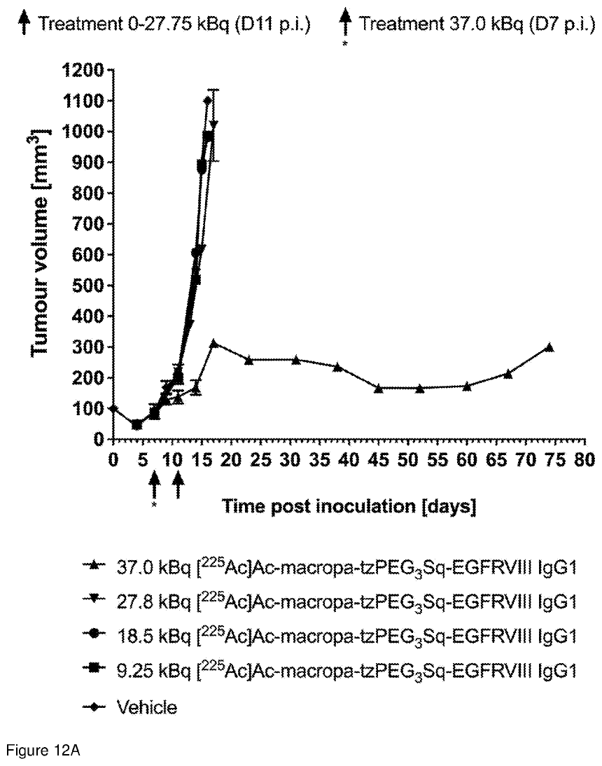

Female, 4-6 weeks old BALB/c nu/nu mice (Animal Research Centre, Western Australia, Perth, Australia) were inoculated with U251 cells (5.5×10\hat{ }cells) in PBS to create subcutaneous tumours in the left flank. At 11 or 14 days post inoculation mice (n=5) bearing approximately 100 mm³ tumour xenografts were injected intravenously with [²²⁵Ac]Ac-macropa-tzPEG₃Sq-EphA3 IgG1 (0.25-1.0 μg/9.25-37.0 kBq) or vehicle PBS. Tumour volume (TV) in mm³ was measured at least biweekly and calculated using the following formula: TV=(L×W²)/2. Data was expressed as mean tumour volume±SD. Statistical analysis was performed at given time points using unpaired t-test. Mice were humanely euthanised at ethical endpoint (TV>1000 mm³ or body weight loss>10%). Results of tumour growth curves and survival data are presented in FIG. 11A and FIG. 11B, respectively. This research project was approved by the Animal Ethics Committee of Austin Health.

Following a single dose of [²²⁵Ac]Ac-macropa-tzPEG₃Sq-EphA3 IgG1, a marked dose response effect was observed for tumour growth inhibition within a few days of treatment. At Day 34 p.i. when vehicle control arm reached ethical endpoint of tumour volume, all treatment arms were significantly different to control (P≤0.0002). The duration of anti-tumour efficacy was also dose dependent. The impact of radiotoxicity was demonstrated through body weight loss. No radiotoxicity was observed at doses up to 18.5 kBq. At the highest dose of 37 kBq radiotoxicity in the form of acute body weight loss was observed within a few days post treatment in 60% of the cohort. At 27.8 kBq radiotoxicity was evident in 20% of cohort by 14 days post treatment. A dose of 18.5 kBq/0.5 μg [²²⁵Ac]Ac-macropa-tzPEG₃Sq-EphA3 IgG1 was determined as the maximum tolerated dose.

### Example 24—Dose Escalation Studies of [²²⁵Ac]Ac-Macropa-tzPEG₃Sq-EGFRVIII IgG1 in Mice Bearing U87MG.de2-7 Xenografts

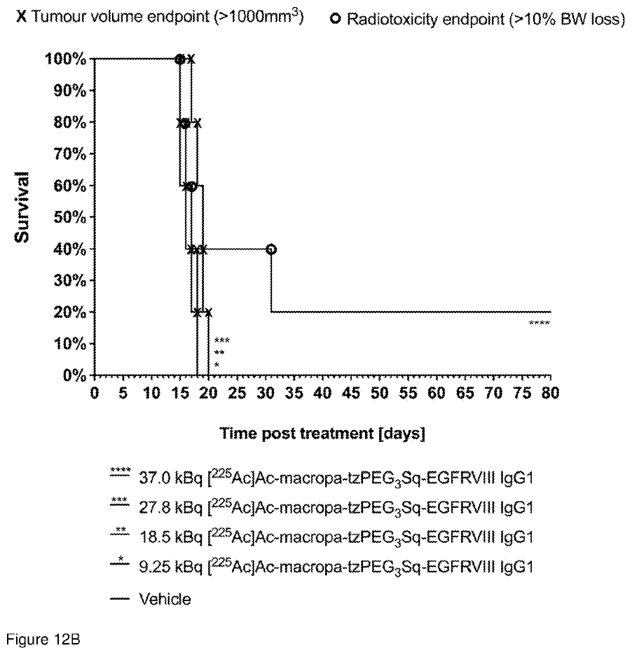

Female, 4-6 weeks old BALB/c nu/nu mice (Animal Research Centre, Western Australia, Perth, Australia) were inoculated with U87MG.de2-7 cells (2.2×10\hat{ }cells) to create subcutaneous tumours in the left flank. At 7 or 11 days post inoculation mice (n=5/group) bearing approximately 100 mm³ tumour xenografts were injected intravenously with [²²⁵Ac]Ac-macropa-tzPEG₃Sq-EGFRVIII IgG1 (0.25-1.0 μg/9.25-37.0 kBq) or vehicle PBS. Tumour volume (TV) in mm³ was measured biweekly and calculated using the following formula: TV=(L×W²)/2. Data was expressed as mean tumour volume±SD. Mice were humanely euthanised at ethical endpoint (TV>1000 mm³ or body weight loss>10%). Results of tumour growth curves and survival data are presented in FIG. 12A and FIG. 12B, respectively. This research project was approved by the Animal Ethics Committee of Austin Health.

The rapidly growing U87MG.de2-7 glioblastoma xenografts precluded anti-tumour efficacy dose response to be observed with only the highest dose of 37 kBq demonstrating prolonged retardation of tumour growth over the duration of the study in 40% of the cohort with radiotoxicity again observed in 60% of cohort within 2 weeks of single dose treatment. A dose of 18.5 kBq/0.5 μg [²²⁵Ac]Ac-macropa-tzPEG₃Sq-EGFRVIII IgG1 was determined as the maximum tolerated dose.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It is to be noted that where a range of values is expressed, it will be clearly understood that this range encompasses the upper and lower limits of the range, and all numerical values or sub-ranges in between these limits as if each numerical value and sub-range is explicitly recited. The statement “about X % to Y %” has the same meaning as “about X % to about Y %,” unless indicated otherwise.

The term “about” as used in the specification means approximately or nearly and in the context of a numerical value or range set forth herein is meant to encompass variations of +/−10% or less, +/−5% or less, +/−1% or less, or +/−0.1% or less of and from the numerical value or range recited or claimed.

It is also to be noted that, as used herein, the singular forms “a”, “an” and “the” include plural aspects unless the context already dictates otherwise.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Future patent applications may be filed in Australia or overseas on the basis of the present application, for example by claiming priority from the present application, by claiming a divisional status and/or by claiming a continuation status. It is to be understood that the following provisional claims are provided by way of example only and are not intended to limit the scope of what may be claimed in any such future application. Furthermore, the claims should not be considered to limit the understanding of (or exclude other understandings of) the invention inherent in the present disclosure. Features may be added to or omitted from the provisional claims at a later date, so as to further define the invention.

1. A compound of the Formula (I) 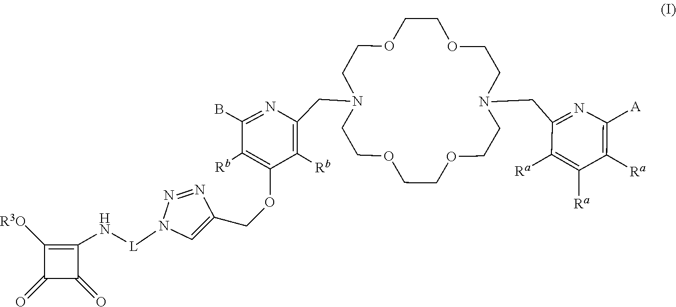
wherein
A is selected from the group consisting of CO₂R¹, and PO₃R¹;
B is selected from the group consisting of CO₂R², and PO₃R²;
R¹, R² and R³ are each independently selected from the group consisting of H and C₁-C₁₂alkyl;
each R^{a} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
each R^{b} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
L is a linker having from 1 to 20 atoms in the normal chain;
or a pharmaceutically acceptable salt thereof.

2. A compound according to claim 1 wherein A is CO₂R¹.

3. A compound according to claim 1 or 2 wherein B is CO₂R².

4. A compound according to any one of claims 1 to 3 wherein R¹ is H.

5. A compound according to any one of claims 1 to 4 wherein R² is H.

6. A compound according to any one of claims 1 to 5 wherein each R^{a} is H.

7. A compound according to any one of claims 1 to 6 wherein each R^{b} is H.

8. A compound according to any one of claims 1 to 7 wherein R³ is C₁-C₁₂alkyl.

9. A compound according to any one of claims 1 to 8 wherein R³ is selected from the group consisting of methyl, ethyl, propyl and butyl.

10. A compound according to any one of claims 1 to 9 wherein L is a linker of the formula:
—(CH₂CH₂O)ₙ—CH₂CH₂—
wherein n is an integer from the group consisting of 0, 1, 2, 3, 4, and 5.

11. A compound according to claim 10 wherein n is 3.

12. A compound of the Formula (Ia): 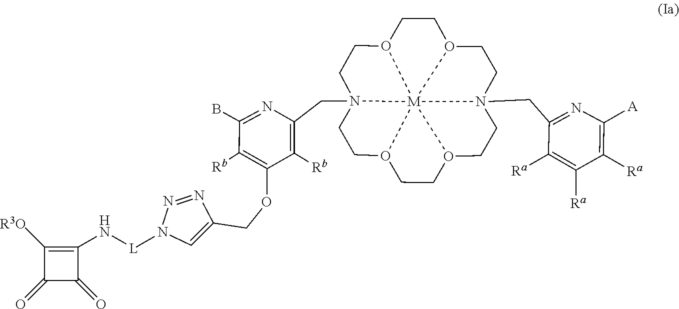
wherein
A is selected from the group consisting of CO₂R¹, and PO₃R¹;
B is selected from the group consisting of CO₂R², and PO₃R²;
R¹, R² and R³ are each independently selected from the group consisting of H and C₁-C₁₂alkyl;
each R^{a} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
each R^{b} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
L is a linker having from 1 to 20 atoms in the normal chain;
M is a radionuclide;
or a pharmaceutically acceptable salt thereof.

13. A compound according to claim 12 wherein A is CO₂R¹.

14. A compound according to claim 12 or 13 wherein B is CO₂R².

15. A compound according to any one of claims 12 to 14 wherein R¹ is H.

16. A compound according to any one of claims 12 to 15 wherein R² is H.

17. A compound according to any one of claims 12 to 16 wherein each R^{a} is H.

18. A compound according to any one of claims 12 to 17 wherein each R^{b} is H.

19. A compound according to any one of claims 12 to 18 wherein R³ is C₁-C₁₂alkyl.

20. A compound according to any one of claims 12 to 19 wherein R³ is selected from the group consisting of methyl, ethyl, propyl and butyl.

21. A compound according to any one of claims 12 to 20 wherein L is a linker of the formula:
—(CH₂CH₂O)ₙ—CH₂CH₂—
wherein n is an integer from the group consisting of 0, 1, 2, 3, 4, and 5.

22. A compound according to claim 21 wherein n is 3.

23. A compound according to any one of claims 12 to 22 wherein the radionuclide is selected from the group consisting of Actinium-225, Lutetium-177, Zirconium-89, Terbium-149, Terbium-152, Terbium-155, Terbium-161, Radium-223, Bismuth-212, Indium-111, Yttrium-86, Yttrium-89, Yttrium-90, and Lead-212.

24. A compound according to any one of claims 12 to 23 wherein M is Actinium-225.

25. A compound of the Formula (Ib): 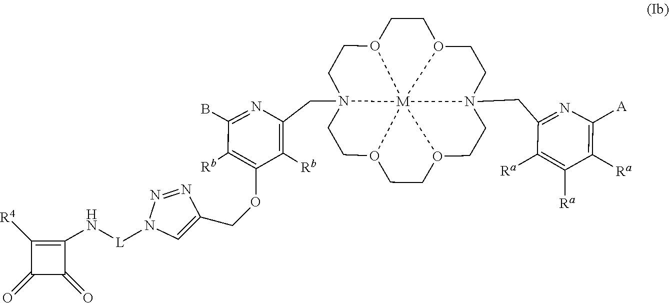
wherein
A is selected from the group consisting of CO₂R¹, and PO₃R¹;
B is selected from the group consisting of CO₂R², and PO₃R²;
R¹, and R² are each independently selected from the group consisting of H and C₁-C₁₂alkyl;
R⁴ is a monoclonal antibody;
each R^{a} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
each R^{b} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
L is a linker having from 1 to 20 atoms in the normal chain;
M is a radionuclide;
or a pharmaceutically acceptable salt thereof.

26. A compound according to claim 25 wherein A is CO₂R¹.

27. A compound according to claim 25 or 26 wherein B is CO₂R².

28. A compound according to any one of claims 25 to 27 wherein R¹ is H.

29. A compound according to any one of claims 25 to 28 wherein R² is H.

30. A compound according to any one of claims 25 to 29 wherein each R^{a} is H.

31. A compound according to any one of claims 25 to 30 wherein each R^{b} is H.

32. A compound according to any one of claims 25 to 31 wherein L is a linker of the formula:
—(CH₂CH₂O)ₙ—CH₂CH₂—
wherein n is an integer from the group consisting of 0, 1, 2, 3, 4, and 5.

33. A compound according to claim 32 wherein n is 3.

34. A compound according to any one of claims 25 to 33 wherein the radionuclide is selected from the group consisting of Actinium-225, Lutetium-177, Zirconium-89, Terbium-149, Terbium-152, Terbium-155, Terbium-161, Radium-223, Bismuth-212, Indium-111, Yttrium-86, Yttrium-89, Yttrium-90, and Lead-212.

35. A compound according to any one of claims 25 to 34 wherein M is Actinium-225.

36. A compound according to any one of claims 25 to 35 wherein the monoclonal antibody is selected from the group consisting of Penpulimab, Sintilimab, Toripalimab, Omburtamab, Tisotumab, Retifanlimab, Ublituximab, Anifrolumab, Loncastuximab, Balstilimab, Dostarlimab, Oportuzumab, Margetuximab, Naxitamab, Belantamab, Tafasitamab, Sacituzumab, Isatuximab, Trastuzumab (Herceptin), Girentuximab, Ifabotuzumab, Depatuxizimab, Enfortumab, Polatuzumab, Emapalumab, Cemiplimab, Moxetumomab, Mogamulizumab, Durvalumab, Avelumab, Atezolizumab, Olaratumab, Daratumumab, Elotuzumab, Necitumumab, Dinutuximab, Nivolumab, Blinatumomab, Blinatumomab, Ramucirumab, Obinutuzumab, Ado-trastuzumab, Pertuzumab, Brentuximab, Ipilimumab, Ofatumumab, Catumaxomab, Panitumumab, Bevacizumab, Cetuximab, Ibritumomab, Alemtuzumab, Gemtuzumab, Rituximab, Edrecolomab, Nimotuzumab, Prolgolimab, and Cetuximab.

37. A pharmaceutical composition comprising a compound according to any one of claims 25 to 36 and a pharmaceutically acceptable carrier.

38. A method of treating a subject the method comprising administering an effective amount of a compound according to any one of claims 25 to 36 to the subject.

39. A method according to claim 38 wherein the subject has cancer.

40. A method for the synthesis of a compound of formula (I): 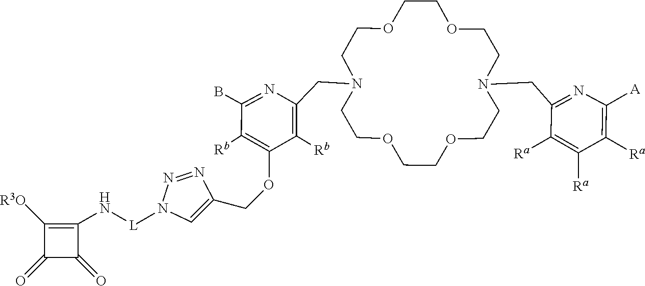
wherein
A is selected from the group consisting of CO₂R¹, and PO₃R¹;
B is selected from the group consisting of CO₂R², and PO₃R²;
R¹, R² and R³ are each independently selected from the group consisting of H and C₁-C₁₂alkyl;
each R^{a} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
each R^{b} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
L is a linker having from 1 to 20 atoms in the normal chain;
or a pharmaceutically acceptable salt thereof;
the method comprising:
(a) providing a compound of formula (10): 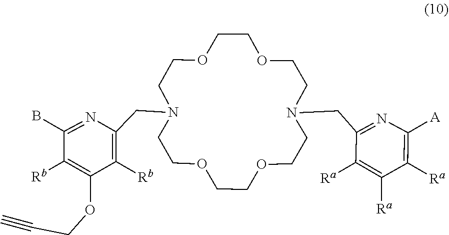
A is selected from the group consisting of CO₂R¹, and PO₃R¹;
B is selected from the group consisting of CO₂R², and PO₃R²;
R¹ and R² are each independently selected from the group consisting of H and C₁-C₁₂alkyl;
each R^{a} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
each R^{b} is independently selected from the group consisting of H, F, Cl, Br, I, CH₃, CH₂CH₃, CH(CH₃)₂, OH, OCH₃, OCH₂CH₃, CF₃, OCF₃, NO₂, NH₂, and CN;
(b) reacting the compound of formula (10) with a compound of formula (11): 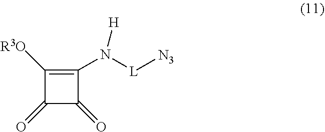
where R³ is selected from the group consisting of H and C₁-C₁₂alkyl;
L is a linker having from 1 to 20 atoms in the normal chain;
in the presence of a copper(I) catalyst.

41. A method according to claim 40 wherein A is CO₂R¹.

42. A method according to claim 40 or 41 wherein B is CO₂R².

43. A method according to any one of claims 40 to 42 wherein R¹ is H.

44. A method according to any one of claims 40 to 43 wherein R² is H.

45. A method according to any one of claims 40 to 44 wherein each R^{a} is H.

46. A method according to any one of claims 40 to 45 wherein each R^{b} is H.

47. A method according to any one of claims 40 to 46 wherein R³ is C₁-C₁₂alkyl.

48. A method according to any one of claims 40 to 47 wherein R³ is selected from the group consisting of methyl, ethyl, propyl and butyl.

49. A method according to any one of claims 40 to 48 wherein L is a linker of the formula:
—(CH₂CH₂O)ₙ—CH₂CH₂—
wherein n is an integer from the group consisting of 0, 1, 2, 3, 4, and 5.

50. A method according to claim 49 wherein n is 3.

51. A method according to any one of claims 40 to 50 wherein step (B) is carried out in the presence of a copper(I) ligand.

52. A method according to claim 51 wherein the copper(I) ligand is selected from the group consisting of TBTA, TEOTA, THPTA, BTTES, BTTAA, BTTP, BTTPS, (BimH)₃, (Bth)₃, BPS, and 4.4′-dimethy-2,2′-bypyrimidine.
